SEQUENCE LISTING (7 sequences)

SEQ ID NO: 1 - DNA, 21 bp, Artificial Sequence
  anti-Triobp siRNA
ccaagcccacaacgaucgutt

SEQ ID NO: 2 - DNA, 21 bp, Artificial Sequence
  anti-Triobp siRNA
acgaucguugugggcuuggtt

SEQ ID NO: 3 - DNA, 36 bp, Artificial Sequence
  mouse Hprt-targeting crRNA
acctcttaggagtctaaagtgttttagagctatgct

SEQ ID NO: 4 - DNA, 20 bp, Artificial Sequence
  FIP-CF568
tcagttcaggaccctcggct

SEQ ID NO: 5 - DNA, 20 bp, Artificial Sequence
  Q2V1
agccgagggtcctgaactga

SEQ ID NO: 6 - DNA, 22 bp, Artificial Sequence
  forward primer
aggtttcgagccctgatattcg

SEQ ID NO: 7 - DNA, 22 bp, Artificial Sequence
  reverse primer
atgtggcaaggtcaaaaacagt

(19) United States
(12) Patent Application Publication
(10) Pub. No.: US 2023/0212611 A1
(43) Pub. Date: Jul. 6, 2023

(54) NANOTUBE MEDIATED DELIVERY SYSTEM AND METHODS COMPRISING THE SAME

(71) Applicants: MONASH UNIVERSITY, Clayton, Victoria (AU); ULVAC, INC., Chigasaki, Kanagawa (JP)

(72) Inventors: Nicolas Hans VOELCKER, Clayton, Victoria (AU); Roey ELNATHAN, Clayton, Victoria (AU); Stella ASLANOGLOU, Clayton, Victoria (AU); Yaping CHEN, Clayton, Victoria (AU)

(21) Appl. No.: 17/798,836

(22) PCT Filed: Feb. 10, 2021
(86) PCT No.: PCT/AU2021/050113

§ 371 Date: Aug. 10, 2022

(30) Foreign Application Priority Data
Feb. 10, 2020 (AU) 2020900357

(51) Int. Cl. C12N 15/89; B82Y 5/00; B82Y 40/00

(52) U.S. Cl. CPC C12N 15/89; B82Y 5/00; B82Y 40/00

(57) ABSTRACT

The present disclosure relates generally to a molecular delivery system and methods of using the molecular delivery system to deliver biomolecules into cells. In particular, the molecular delivery system comprises a nanotube (NT) array comprising a plurality of nanotubes (NTs) which are used to deliver biomolecules to cells. The NTs can be loaded with molecules that can be delivered into cells following contact (e.g. penetration) by the NTs.

### FIELD OF THE INVENTION

The present disclosure relates generally to a molecular delivery system and methods of using the molecular delivery system to deliver biomolecules into cells. In particular, the molecular delivery system comprises a nanotube (NT) array comprising a plurality of nanotubes (NTs).

### BACKGROUND OF THE INVENTION

The delivery of biological material such as small molecules, proteins, and genetic material to cells, for example across the cell membrane barrier and into the cell interior, is a critical step for molecular biology and ex-vivo gene and cell therapy. Intracellular delivery of biomolecules to cells can be achieved using viral (for example, adenoviruses, retroviruses, adeno-associated viruses, herpes simplex viruses and vaccinia viruses) and chemical (for example, lipofection, calcium-phosphate, DEA-dextran) methods. Many of these methods are hampered by lysosomal degradation, cell-type specificity, low efficiency, expense and/or toxicity concerns.

Physical methods for disrupting cellular membranes so as to allow for the delivery of biologicals into the cell interior include direct penetration modalities and permeabilization. Bulk electroporation (EP) is a popular permeabilization technique that makes the cell more permeable by the application of a potential difference across that membrane which causes the lipid biomolecules within the membrane to re-orient to form small hydrophilic openings on the cell membrane and thus permit passage of biological through the openings. Despite its widespread adoption some challenges however remain, such as requiring high voltages (e.g. kV range), joule heating at the solution-electrode interface and/or lack of precise dose control and variable cell viability.

These limitations have led to an increased effort to develop alternative physical approaches, including microinjection processes to physically pump biologicals into cells via microfluidic integration. Some research is emerging using nanostraws (i.e. small hollow tubes), with one opening at a reservoir containing the biological material, and the other opening presenting to the cell suspension which penetrates into the cell interior. The biological material flows from the reservoir through the hollow straws and into penetrated cells, often facilitated by small electrical currents or microfluidic pumps. Currently these nanoscale, and microfluidic integrated methods are cumbersome, complex systems to design and make at scale and difficult to combine with tissue culture ware for routine experiments, and can result in significant waste of biological materials, labour-intensive handling and lack of reproducibility and precision.

Accordingly, there is a need to develop efficient, minimally invasive approaches for delivering biologicals to cells.

### SUMMARY OF THE INVENTION

The present inventors have developed a method for delivering a molecule to a cell using a nanotube (NT) array loaded with a molecule. The molecule may be a biomolecule. The NT array comprises a base and a plurality of nanotubes (NTs) attached at one end to the base. The base may be a solid base. The base may be a solid impermeable base. The plurality of NTs extend from the base, for example, the NTs are upstanding from the base, and extend away from the attachment point on the base. Each NT comprises a wall defining a single opening. The single opening is at the other end of the NT (i.e. the end not attached to the base). The wall and single opening together provide an inner cavity which can be configured to host biomolecules. The inner cavity can be loaded with a solution comprising biomolecules. To deliver biomolecules to a cell, cells are incubated with the NT array. One or more NTs contact the cell, and the biomolecule can be transferred from the inner cavity of the NT to the cell, for example via mechanical penetration, endocytosis, and/or membrane permeabilisation.

In one aspect, there is provided a method of delivering a biomolecule to a cell, the method comprising:
a) combining cells with a nanotube (NT) array, comprising:
   i) a solid impermeable base; and
   ii) a plurality of nanotubes (NTs) attached at one end to the solid impermeable base;
   wherein each NT comprises a wall defining a single opening at the other end of the NT to provide an inner cavity, and
   the inner cavity of at least some of the NTs is loaded with a solution comprising the biomolecule, and
b) incubating the cells with the NT array to deliver the biomolecule to at least some of the cells.

In one embodiment, the cells are incubated with the NT array to deliver the biomolecule from the inner cavity of at least some of the NTs to at least some of the cells.

In one embodiment, the method comprises co-delivering two or more biomolecules to a cell, wherein the inner cavity is loaded with a solution comprising two or more biomolecules to co-deliver the biomolecules to the cell.

In another or further embodiment, the method further comprises loading a solution comprising the biomolecule(s) into the inner cavity of at least some of the NTs prior to combining the cells with the NT array at step a). In one embodiment, the NTs are incubated with the biomolecule(s) for about 30 minutes to about 6 h, for example about 30 minutes to about 2 h.

In one embodiment, the biomolecule(s) are selected from one or more of a nucleic acid (e.g. a therapeutic nucleic acid), a protein, a peptide, a polysaccharide, or a small biomolecule, or a combination thereof. The biomolecule(s) may be capable of editing the genome of the cell. The biomolecule(s) may be selected from one or more of a DNA, RNA, or siRNA biomolecule, or a protein, or a combination thereof. The nucleic acid may be a plasmid, dsDNA, mRNA, miRNA, PNA, or siRNA, or a combination thereof. The nucleic acid may encode a chimeric antigen receptor (CAR). The protein may be an antibody or an enzyme, including for example a programmable nuclease. The programmable nuclease may be capable of editing the genome of the cell.

In one embodiment, the loading concentration of the biomolecule in the solution is between about 100 µg.mL⁻¹ to about 2,000 µg.mL⁻¹.

In one embodiment, the cell is an adherent, non-adherent, immortalised, primary cell or stem cell. In one embodiment, the cell is a mammalian cell. The immortalised cell or primary cell may be an immune cell, neuron, endothelial cell, epithelial cell, or fibroblast. The immune cell may be a T cell, B cell, dendritic cell, macrophage, or natural killer cell. The stem cell may be an embryonic hematopoietic, mesenchymal, or induced pluripotent stem cell.

In one embodiment, the cells are incubated with the NT array for about 1 h to about 24 h. In another or further embodiment, the cells are incubated with the NT array at a temperature of about 20° C. to about 40° C. In another or further embodiment, the method comprises the step of centrifuging the cells and NT array during the incubation at step b). In another or further embodiment, the method comprises the step of detaching the cells from the NTs after the incubation at step b). In another or further embodiment, the method comprises the step of culturing the cells.

In one embodiment, the NTs have an average density of between about 0.1 to about 4.0 NTs per µm². In another or further embodiment, the NTs have an average length of between about 1 µm to about 5 µm. In another or further embodiment, the NTs have an average inner cavity diameter of between about 50 nm to about 500 nm. In another or further embodiment, the NTs have an average wall thickness of between about 20 nm to about 200 nm. In one embodiment, the NTs have i) an average density of between about 0.1 to about 4.0 NTs per µm²; ii) an average length of between about 1 µm to about 5 µm; iii) an average inner cavity diameter of between about 50 nm to about 500 nm; and iv) an average wall thickness of between about 20 nm to about 200 nm.

In one embodiment, the NTs have an average density of between about 0.1 to about 0.5 NTs per µm². In another or further embodiment, the NTs have an average length of between about 2 µm to about 5 µm. In another or further embodiment, the NTs have an average inner cavity diameter of between about 200 nm to about 500 nm. In another or further embodiment, the NTs have an average wall thickness of between about 50 nm to about 200 nm. In another or further embodiment, the NTs have an average pitch of between about 0.1 µm to about 10 µm. The NTs may have a combination of any two or more of the average densities, lengths, inner cavity diameters, pitch as described herein. In one embodiment, the NTs have a combination of any two or more of: i) an average density of between about 0.1 to about 0.5 NTs per µm²; ii) an average length of between about 2 µm to about 5 µm; iii) an average inner cavity diameter of between about 200 nm to about 500 nm; iv) an average wall thickness of between about 50 nm to about 200 nm; and v) an average pitch of between about 0.1 µm to about 10 µm.

In one embodiment, the NTs are silicon NTs, polymeric NTs, or a combination thereof. In one embodiment, the polymeric NTs made from polystyrene, polyesters, polycarbonates, polypyrroles, hybrid ceramic based polymers or epoxy based photoresists, or a combination thereof. The polymeric NTs may be made from polystyrene, polyesters such as Thermanox™, polycarbonates, polypyrroles, hybrid ceramic based polymers such as silanes e.g. Ormocers (ORMOCOMP), or epoxy based resists such as SU8, or a combination thereof.

In one embodiment, the NTs may be silicon NTs. In one embodiment, the silicon NTs have an average density of between about 0.1 to about 0.5 NTs per µm². In another or further embodiment, the silicon NTs have an average length of between about 2 µm to about 5 µm. In one embodiment, the silicon NTs have an average inner cavity diameter of between about 200 nm to about 500 nm. In another or further embodiment, the silicon NTs have an average wall thickness of between about 50 nm to about 200 nm. In another or further embodiment, the silicon NTs have an average pitch of between about 0.1 µm to about 10 µm. The silicon NTs may have a combination of any two or more of the average densities, lengths, inner cavity diameters, pitch as described herein. In one embodiment, the silicon NTs have a combination of any two or more of: i) an average density of between about 0.1 to about 0.5 NTs per µm²; ii) an average length of between about 2 µm to about 5 µm; iii) an average inner cavity diameter of between about 200 nm to about 500 nm; iv) an average wall thickness of between about 50 nm to about 200 nm; and v) an average pitch of between about 0.1 µm to about 10 µm.

In one embodiment, the NTs may be polymeric NTs (i.e. are made from a polymeric material). In one embodiment, the polymeric NTs have an average density of between about 0.1 to about 0.5 NTs per µm². In another or further embodiment, the polymeric NTs have an average length of between about 2 µm to about 5 µm. In one embodiment, the polystyrene NTs have an average inner cavity diameter of between about 200 nm to about 500 nm. In another or further embodiment, the polymeric NTs have an average wall thickness of between about 50 nm to about 200 nm. In another or further embodiment, the polymeric NTs have an average pitch of between about 0.1 µm to about 10 µm. The polymeric NTs may have a combination of any two or more of the average densities, lengths, inner cavity diameters, pitch as described herein. In one embodiment, the polymeric NTs have a combination of any two or more of: i) an average density of between about 0.1 to about 0.5 NTs per µm²; ii) an average length of between about 2 µm to about 5 µm; iii) an average inner cavity diameter of between about 200 nm to about 500 nm; iv) an average wall thickness of between about 50 nm to about 200 nm; and v) an average pitch of between about 0.1 µm to about 10 µm.

In one embodiment, the NTs may be polystyrene NTs. In one embodiment, the polystyrene NTs have an average density of between about 0.1 to about 0.5 NTs per µm². In another or further embodiment, the polystyrene NTs have an average length of between about 2 µm to about 5 µm. In one embodiment, the polystyrene NTs have an average inner cavity diameter of between about 200 nm to about 500 nm. In another or further embodiment, the polystyrene NTs have an average wall thickness of between about 50 nm to about 200 nm. In another or further embodiment, the polystyrene NTs have an average pitch of between about 0.1 µm to about 10 µm. The polystyrene NTs may have a combination of any two or more of the average densities, lengths, inner cavity diameters, pitch as described herein. In one embodiment, the polystyrene NTs have a combination of any two or more of: i) an average density of between about 0.1 to about 0.5 NTs per µm²; ii) an average length of between about 2 µm to about 5 µm; iii) an average inner cavity diameter of between about 200 nm to about 500 nm; iv) an average wall thickness of between about 50 nm to about 200 nm; and v) an average pitch of between about 0.1 µm to about 10 µm.

In one embodiment, the plurality of NTs extend substantially vertically from the solid impermeable base.

In a related and second aspect, there is provided a nanotube (NT) array for delivering a biomolecule to a cell, the NT array comprising:
i) a solid impermeable base; and
ii) a plurality of nanotubes (NTs) attached at one end to the solid impermeable base;
wherein each NT comprises a wall defining a single opening at the other end of the NT to provide an inner cavity.

In one embodiment, the inner cavity of at least some of the NTs is loaded with a solution comprising the biomolecule. In another embodiment, the NTs have an average density of between about 0.1 to about 0.5 NTs per µm².

In a related and third aspect, there is provided a population of cells comprising a biomolecule, and/or which have been modified by the biomolecule, the biomolecule having been delivered to the population of cells, or progenitors thereof, by application and incubation on NT arrays.

In one embodiment, the population of cells are applied and incubated with a NT array according to any one of the second or third aspects. In one embodiment, the population of cells is delivered with a biomolecule using the method according to the first aspect.

In one embodiment, the population of cells are primary immune cells. The cells may comprise CAR⁺ T cells. The cells may not be activated prior to delivery of the biomolecule.

In an embodiment, the biomolecule is a programmable nuclease.

In a related and fourth aspect, there is provided a kit for delivering a biomolecule to a cell, comprising:
a) a NT array as described above; and
b) a vessel configured to house the NT array.

In one embodiment, the vessel is a multiwell plate, a petri dish or a flask. In another or further embodiment, the NT array forms the base of the vessel. In one embodiment, the kit further comprises c) a solution comprising the biomolecule. In another or further embodiment, the kit further comprises d) a cell suspension. It will be appreciated that embodiments described for the NTs, biomolecules, and/or cells earlier in relation the NT array and method of delivering a biomolecule to a cell can also provide embodiments for the NTs, biomolecules and cells that are part of the kit.

In a related and fifth aspect, there is provided method of delivering a biomolecule to a cell, the method comprising:
a) combining a cell suspension containing cells with a nanotube (NT) attached at one end to the solid impermeable base,
   wherein the NT comprises a wall defining a single opening at the other end of the NT to provide an inner cavity, and
   the inner cavity of the NT is loaded with a solution comprising the biomolecule, and
b) incubating the cell suspension with the NT to deliver the biomolecule to the cells in the cell suspension.

In a related and sixth aspect, there is provided a nanotube (NT) attached at one end to a solid impermeable base for delivering a biomolecule to a cell, wherein the NT comprises a wall defining a single opening at the other end of the NT to provide an inner cavity.

In one embodiment, the inner cavity of the NT is loaded with a solution comprising the biomolecule. In another embodiment, the NT has an inner cavity diameter of between about 200 nm to about 500 nm.

In a related aspect, there is provided a nanotube (NT) array for delivering a biomolecule to a cell, the NT array comprising: i) a solid impermeable base; and ii) a plurality of nanotubes (NTs) attached at one end to the solid impermeable base; wherein each NT comprises a wall defining a single opening at the other end of the NT to provide an inner cavity, and the inner cavity of at least some of the NTs is loaded with a solution comprising the biomolecule.

In a related aspect, there is provided a nanotube (NT) array for delivering a biomolecule to a cell, the NT array comprising: i) a solid impermeable base; and ii) a plurality of nanotubes (NTs) attached at one end to the solid impermeable base; wherein each NT comprises a wall defining a single opening at the other end of the NT to provide an inner cavity, and the NTs have an average density of between about 0.1 to about 0.5 NTs per µm².

In a related aspect, there is provided a nanotube (NT) attached at one end to a solid impermeable base for delivering a biomolecule to a cell, wherein the NT comprises a wall defining a single opening at the other end of the NT to provide an inner cavity, and the inner cavity of the NT is loaded with a solution comprising the biomolecule.

In a related aspect, there is provided a nanotube NT attached at one end to a solid impermeable base for delivering a biomolecule to a cell, wherein the NT comprises a wall defining a single opening at the other end of the NT to provide an inner cavity, and the NT has an inner cavity diameter of between about 200 nm to about 500 nm.

It will be appreciated that embodiments described for the NTs, biomolecules, and/or cells earlier in relation the method of delivering a biomolecule to a cell can also provide embodiments for the NT array, NTs, kits and population of cells described herein, and vice versa.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF FIGURES

FIG. 1 - Fabricated silicon nanotube (SiNT) arrays comprise vertically aligned NTs having an internally defined cavity: SEM images showing the SiNTs at tilted zoom-out (i) and zoom-in (ii), top (iii), and vertical (resin embedded) view (iv). Scale bars, 5 µm (i), 500 nm (ii), and 300 nm (iii, iv).

FIG. 2 - SiNT arrays loaded with biomolecules: Confocal images showing the top (i-iii) and 3D views (iv) of SiNTs loaded with IgG-AF488 (i, iv), IgG-AF647 (ii), and ssDNA-FAM (iii), respectively. Insets (i-iii) are 5× enlargements. Scale bars, 20 µm (i-iii) and 5 µm (iv).

FIG. 3 - Payload concentration of biomolecules was optimized for maximum SiNT loading and release: (a-b) Representative confocal microscopy images showing SiNTs loaded with (a) IgG-AF647 (conc. at 2, 20, 200, and 2000 µg mL⁻¹) and (b) ssDNA-FAM (conc. at 1.6, 16, 160, and 1600 µg mL⁻¹). Scale bars, 40 µm. Insets are 8 × enlargements of the main images. (c-d) Statistical analysis of the mean fluorescence intensity (arbitrary unit, a.u.) of AF647 and FAM across SiNT substrates loaded with (c) IgG-AF647 (conc. between 0.2-2000 µg mL⁻¹) and (d) ssDNA-FAM (conc. between 0.16-1600 µg mL⁻¹). (e) Statistical analysis of the mean fluorescence intensity (a.u.) of the release of AF647 on the same SiNT substrate at 6, 24, and 48 h after loading with IgG-AF647 (conc. at 200 µg mL⁻¹).

FIG. 4 - SiNTs mediate delivery of ssDNA into cells: (a) Max projection of confocal z-stack images demonstrating GPE86 cells after 6 h culture on flat Si (i) and SiNT (ii) substrates that are pre-loaded with ssDNA-FAM. Cells were fixed and stained with Hoechst and phalloidin. Scale bars, 10 µm. (iii)-(iv) show the FAM intensity (arbitrary unit, a.u.) of the corresponding area (dashed line) in the main figures (i,ii). (b) Quantification of the percentage of cells containing ssDNA-FAM on flat Si and SiNT substrates. ***P = 0.0005 (Mann-Whitney’s U-tests).

FIG. 5 - SiNTs mediate delivery of IgGs into cells: (a) Max projection of confocal z-stack images demonstrating GPE86 cells after 6 h culture on (i) flat Si and (ii) SiNT substrates that were pre-loaded with IgG-AF647. Cells were fixed and stained with Hoechst and phalloidin. Scale bars, 10 µm. (b) Quantification of the percentage of cells containing IgG-AF647 on flat Si and SiNT substrates. ***P = 0.0005 (Mann-Whitney’s U-tests).

FIG. 6 - Biomolecules delivered by SiNTs gain direct cytosolic entry: SiNT-mediated delivery of fluorescent internalization probe (FIP) into GPE86 cells. (a-b) Max projection of confocal z-stack images showing the distribution of CF568-tagged FIP (FIP-CF568) in cells cultured on SiNTs untreated (a), and treated with (b) Q2V1 quencher that diminishes uninternalized FIP-CF568. Cells were stained with Hoechst 5 min before imaging. Scale bars, 10 µm. (c) Histogram graph showing the overlay of CF568 intensity within control cells (Ctrl) and cells detached from FIP-CF568 coated SiNTs that were treated without or with Q2V1 quencher. (d) Quantification of the relative CF568 GMFI of the three groups of cells described in (c). *P = 0.0151, **P = 0.0015, ***P = 0.0005 (one-way ANOVA).

FIG. 7 - Biomolecules delivered by SiNTs into cells maintain their biological functionality: SiNTs mediate intracellular delivery of Cy5-tagged GFP-encoding mRNAs (Cy5-mRNA-GFP) into GPE86 cells. (a-b) Max projection of confocal z-stack images demonstrating GPE86 cells after 6 h culture on (a) flat Si and (b) SiNT substrates that were pre-loaded with Cy5-mRNA-GFP. Cells were stained with Hoechst 5 minutes before imaging. Scale bars, (i) 40 µm and zoom-in (ii)10 µm. (c) Flow cytometry analysis of GPE86 cells detached from flat Si and SiNT substrates pre-loaded with Cy5-mRNA-GFP. Circle gating indicates Cy5⁺ population. (d) Quantification of the percentage of Cy5⁺ population (left) and the expression of GFP (right) within cells from flat Si and SiNT substrates as in (c). **P = 0.0048, ***P = 0.0004 (Mann-Whitney’s U-tests).

FIG. 8 - SiNTs can be co-loaded with multiple biomolecules: Co-loading of fluorescence-tagged IgGs onto SiNTs. (a) Representative confocal microscopy images showing SiNTs loaded with IgG-AF647 and IgG-AF488 at five different v/v ratios (10:0, 7:3, 5:5, 3:7, 0:10; total volume 10 µL; total IgG conc. 200 µg mL⁻¹). Scale bars, 40 µm. Insets are 8× enlargements of the main images. (b) Statistical analysis of the mean fluorescence intensity (a.u.) of both AF647 and AF488 on SiNT substrates loaded with IgG-AF647 and IgG-AF488 at each v/v ratio. (c) Statistical analysis of the total fluorescence intensity (a.u.) including AF647 and AF488 on IgG-loaded SiNT substrates at each v/v ratio. ***P = 0.0002, ****P < 0.0001 (one-way ANOVA).

FIG. 9 - SiNTs mediate simultaneous delivery of multiple biomolecules into cells: SiNT-mediated co-delivery of fluorescence-tagged IgGs into GPE86 cells. (a-c) Max projection of confocal z-stack images showing GPE86 cells cultured 6 h on SiNTs pre-loaded with IgG-AF647 and IgG-AF488 with v/v ratio at 10:0 (a), 5:5 (b), and 0:10 (c) (total volume 10 µL; total IgG conc. 200 µg mL⁻¹). Cells were fixed and stained with Hoechst and phalloidin. (d-e) Flow cytometry analysis showing the plots gating (d) and their correlated quantification (e) of AF647⁺, AF488⁺, and AF647⁺-AF488⁺ populations within GPE86 cells detached from SiNTs as described in (a-c).

FIG. 10 - SiNTs achieve tight interfacing during incubation with cells: SEM images showing the interfacial interaction between GPE86 cells with SiNTs at tilted (i) and top (ii) views. Insets are enlargements of the areas outlined in the main figures. Scale bars, 10 µm for the main figures (10i, ii) and 2 µm for the insets.

FIG. 11 - Tight interfacing during incubation with SiNTs does not compromise cell viability: Quantification of the viability of GPE86 cells after 6 h culture on flat Si and SiNT substrates loaded with or without IgG-AF647 (n ≥ 3).

FIG. 12 - Tight interfacing during incubation with SiNTs induces localized membrane deformation while maintaining membrane integrity: SEM images after FIB milling at 90° (i) and 45° (ii) demonstrating the membrane invaginations (indicated by arrows) of GPE86 cells induced by SiNTs. Scale bar, 500 nm. Original images are black-white inverted.

FIG. 13 - SiNTs can directly activate endocytic processes in cells: SiNTs induce endocytosis mediated by caveolin-1. Confocal images showing GPE86 cells after 6 h culture on flat Si (a) and SiNT (b) substrates that were pre-loaded with ssDNA-FAM. Cells were fixed and stained with Hoechst, anti-clathrin heavy chain (CHC), and anti-caveolin-1 (CAV-1). (ii) Zoom-in view of the outlined area in (i). Scale bars, 10 µm (a i, b i) and 5 µm (a ii, b ii). Arrows (b ii) indicate the colocalization of CAV-1 and ssDNA-FAM within a GPE86 cells on SiNTs.

FIG. 14 - Caveolae inhibition compromises SiNT-induced endocytosis in cells: Impact of endocytosis inhibition on SiNT-mediated delivery. (a-f) Max projection of confocal z-stack images showing untreated GPE86 cells (a, d), and GPE86 cells treated with Nystatin (b, e) or chlorpromazine (c, f) on SiNTs pre-loaded with Cy5-mRNA-GFP. Cells were stained with Hoechst 10 minutes before imaging. (g-i) Cy5 intensity (arbitrary unit, a.u.) of the corresponding area (dashed line) in the main figures in (d-f).

FIG. 15 - SiNTs enable intracellular siRNA delivery: Cells seeded on SiNTs loaded with anti-Triobp siRNAs show reduced cell attachment and cell count. (a) Large-scan fluorescence images showing the distribution of GPE86 cells after 48 h culture on SiNTs loaded with negative control scramble siRNA (Neg) and triobp-targeting siRNA (anti-Triobp). Cells were stained with Hoechst, anti-Triobp, phalloidin, and WGA. Scale bars, 500 µm. (b) Quantification of the average cell count of GPE86 cells on SiNTs coated with Neg and anti-Triobp siRNAs. ***P = 0.0005 (Mann-Whitney’s U-tests).

FIG. 16 - SiNTs enable intracellular siRNA delivery and siRNA-mediated gene knock-down: Cells seeded on SiNTs loaded with anti-Triobp siRNAs show reduced Triobp expression, localization and actin cytoskeleton formation. Confocal microscopy images show GPE86 cells after 48 h culture on SiNTs that were pre-loaded with negative control scramble (Neg, left) and Triobp-targeting (anti-Triobp, right) siRNAs. Cells were stained with Hoechst, Triobp antibody, phalloidin, and WGA. Scale bars, 20 µm.

FIG. 17 - SiNTs enable intracellular Cas9 RNP delivery: Cells seeded on SiNTs loaded with Cas9-ATTO RNP show transfection with Cas9-ATTO RNP. (a) Flow cytometry plots showing the gating of ATTO 550⁺ GPE86 cells detached from SiNTs loaded with Cas9-ATTO RNP. Untreated GPE86 cells serve as control (ctrl). (b) Quantification of the percentage of ATTO 550⁺ GPE86 cells in both ctrl and Cas9-ATTO groups as in (a). *P = 0.0254 (Mann-Whitney’s U-tests). (c) Confocal microscopy images showing the expression of ATTO 550 within GPE86 cells 24 h after detachment from SiNTs pre-loaded with Cas9:tracrRNA ATTO 550 complex (Cas9-ATTO). Untreated cells served as negative control (ctrl).

FIG. 18 - SiNTs enable intracellular Cas9 RNP delivery and Cas9 RNP-mediated gene editing: Cells seeded on SiNTs loaded with Hprt-targeted Cas9 RNP show Hprt gene cleavage. (a) T7E1 assay of re-annealed PCR products from GPE86 cells transfected with (i) Cas: control-crRNA by SiNTs (SiNT-Neg), or, (ii) transfected with Cas : Hprt-crRNA by SiNTs (SiNT-Hprt) or, (iii) Lipofectamine 2000 (Lipo-Hprt). Cleavage bands are indicated by arrows and DNA ladder size (left column). (b) Calculation of cleavage efficiency based on the result of T7E1 assay in (a). *P = 0.0247, **P = 0.0014 (one-way ANOVA).

FIG. 19 - Optimisation of NT array geometry for different cell types/sizes: (a) Confocal images showing the Hoechst stained primary mouse T cells on SiNTs with pitches of 1 (i), 2 (ii), 3 (iii), and 5 µm (iv), respectively. (b) SEM images showing the fixed primary mouse T cells on SiNTs with pitches of 1 (i), 2 (ii), 3 (iii), and 5 µm (iv), respectively. (c) Confocal images showing the primary mouse T cells after 6 h culture on 3 µm SiNTs pre-loaded with Cy5-mRNA-GFP. Cells were stained with Hoechst 10 minutes before imaging.

FIG. 20 - Schematic summarising a method of delivering a biomolecule to a cell: A schematic of the method of delivering a biomolecule (104) to a cell (101) using a NT array (102 and 103) according to some embodiments of the present disclosure.

FIG. 21- Schematic of a single NT: An enlargement of the circled area (200) of the biomolecule delivery system in FIG. 1, FIG. 24 and FIG. 25 depicting a single NT with a wall (201) and an opening (202) biomolecules (X and Y) loaded in the cavity (203) of the NT. (A and B).

FIG. 22 - Cells can be added onto the NT array which is housed in a vessel: A schematic depicting the adding of cells onto the NT array in a cell plate using an embodiment of the invention, where (300) is the cell plate and (301) is a well of that cell plate.

FIG. 23 - Cells can be added onto the NT array which is housed in a tissue flask: A schematic depicting the incubation of a population of cells on the NT array in a tissue flask.

FIG. 24 - Comparison of SiNT and polymeric NT. (a) Tilted (45°) SEM image of SiNTs fabricated by EBL and DRIE. (b) Close up SEM image of SiNTs of height 2 µm and diameter 500 nm. (c) Tilted (45°) SEM image of SU8 replica fabricated from SiNT templates by NIL. (d) Close up SEM image of the SU8 replica showing a NT structure.

FIG. 25 - SEM images of SU8 NTs with partially opened structure. (a) Tilted (45°) SEM image of SU8 NTs showing some of the tubes are closed or partially opened (marked by arrows). (b) Top-view SEM image of SU8 NTs with tubes that are closed or partially opened (marked by arrows).

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art.

As used herein, the term “about”, unless stated to the contrary, refers to +/- 10%, more preferably +/- 5%, more preferably +/- 1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “NT” and “NTs” are abbreviations of the term nanotube and nanotubes. These terms are used interchangeable throughout the disclosure.

The term “nanotube” or “NT” refers to one or more projections having a cross-sectional diameter in the range of 1 nm to 1 µm, wherein the projection comprises a wall and base, wherein the wall defines an opening at one end of the NT to provide an inner cavity within the NT.

The term “Si” refers to silicon. These terms are used interchangeably throughout the disclosure.

The term “SiNT” or “SiNTs” refers to silicon nanotubes. These terms are used interchangeably throughout the disclosure.

The terms “attached at one end” or “extending from the base” are understood to mean that the plurality of NTs are only attached at one end, or extend from and thus project away from the surface of the base. For example, the proximal ends of the NTs are each attached to the base, and the distal ends of the same NTs (e.g. the ends with the opening) are each located a certain distance away from the base, resulting in the NTs extending away from the surface of the base. The term “single opening” is understood to mean that the NT comprises only one opening in which the interior cavity of the NT can be accessed. It will be appreciated that each NT comprises two ends, where one end is attached to the base and the other end of the NT has a single opening. The end attached to the base and the other end having a single opening are on opposite ends of the NT. The end of the NT attached to the base can be called the proximal end of the NT, and the other end of the NT having a single opening can be called the distal end of the NT. The distal end (e.g. “the other end” of the NT with respect to the end attached to the base) is located a certain distance away from the proximal end (e.g. the end attached to the base), resulting in the NTs extending away from the surface of the base. The end of the NT attached to the base may also be called a first end, and the other end of the NT having a single opening may also be called a second end. The term “distal end” and “the other end”, and “proximal end” are used interchangeably throughout the present disclosure.

The term “biomolecule” is understood to mean any substance or matter that can be delivered to the cell. The term biomolecule also includes two or more biomolecules. For example, the term biomolecule includes, but is not limited to, a protein, a nucleic acid, a nanoparticle, a dye, a toxin, a virus, a peptide, and a polymer particle.

Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Nanotube Arrays and Delivery of Biomolecules to Cells

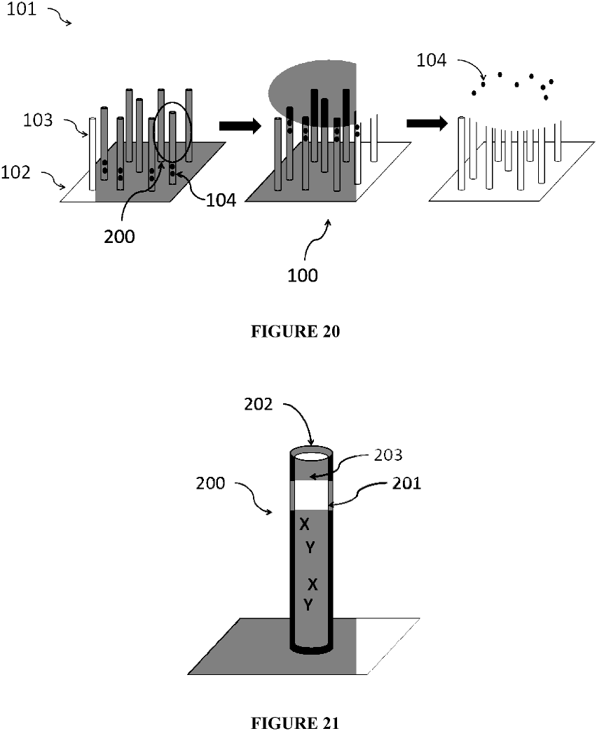

The present disclosure relates to the use of NT arrays to deliver exogenous biomolecules to cells. Referring to FIG. 20A, an example of a method of delivering biomolecules to cells is shown (100). A cell (101) from a cell suspension is brought into contact with a NT array comprising a solid base (e.g. a solid impermeable base) having a surface (102) having a plurality of NTs (103) attached at one end to the base. Some of the NTs (103) comprise one or more biomolecules (104) to be delivered to a cell. Referring to FIG. 21, each of the NTs (200) comprise a wall (201) which defines an opening (202) at the other end of the NT (e.g. the distal end) to provide an inner cavity (103). The inner cavity (103) of at least some of the NTs can be loaded with various biomolecules, for example by loading the inner cavity of at least some of the NTs with a liquid solution comprising one or more biomolecules (104) (e.g. X and/or Y). Suitable biomolecules that can be delivered into cells are described herein. The geometry of the NTs in the NT array (e.g. pitch, density, height, diameter etc.) can vary and is described herein. In an embodiment, the geometry of the array can be programmed based on the type of cell being targeted and/or the type of biomolecule being delivered to the cell. For example, the size of the inner cavity can be tailored to accommodate a wide variety of bioactive biomolecules. Additionally or alternatively, the density of the NTs can be tailored to accommodate various cell sizes.

In FIG. 20A, the NTs (103) loaded with biomolecules (104) can come into contact, and in some embodiments penetrate into cells (101) which have been added onto the NTs, by way of a cell suspension. Following incubation, at least some of the biomolecules (104) may be delivered or co-delivered to at least some of the cells. The introduction of the biomolecules into the cell may alter the cellular function, for example, result in a transformed immune cell (e.g. a CAR⁺ T cell), an edited genome (e.g. by delivering a programmable nuclease, such as a ribonucleoprotein comprising a programmable nuclease, for example Cas9 RNP) or gene silencing (e.g. by RNAi).

### Nanotube Arrays

The method of delivery a biomolecule to a cell comprises adding cells from a cell suspension onto a NT array. The NT array comprises a base and a plurality of NTs attached at one end to the base.

In some embodiments, the NT array comprises a solid impermeable base and a plurality of NTs attached at one end to the solid impermeable base Depending on the orientation of the solid impermeable base in relation to the perspective of an external observer, it will be appreciated that the plurality of NTs attached at one end to the solid impermeable base may be projecting upwards, downwards, or sideways in relation to the base. The NTs may be described as a plurality of upstanding NTs attached to the solid impermeable base. The NTs may be attached to the surface of the solid permeable base along a substantially vertical direction to the surface. In this embodiment, a person skilled in the art would understand the NTs as being “vertically aligned” or “vertically aligned NTs”. In some embodiments, the NTs may form at an angle with respect to the base, for example an angle of between about 60° to about 90°, between about 70° to about 90°, between about 80° to about 90°, for example about 85°, or about 90°. In some embodiments, the NTs average angle with respect to the base is about 90°.

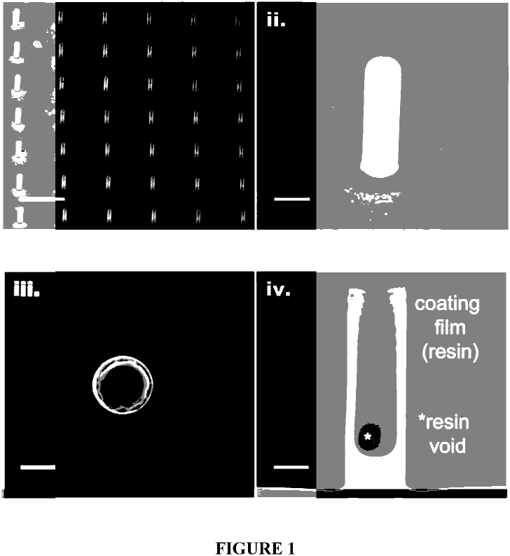

In some embodiments, the solid impermeable base may also be called a substrate. The solid impermeable base is in direct communication with the ends of the plurality of the NTs attached to the base to form a closure at one end of the NTs. For example, the solid impermeable base functions as a closure means at the proximal end (i.e. the end of the NTs attached to the base) of the plurality of NTs, such that the inner cavity of the NTs does not extend through the solid impermeable base and biomolecules cannot flow through the base. The cavity act as a reservoir keeping the biomolecules within the tube and in direct communication with the cells and cell suspension upon incubation, as seen in FIGS. 1 (iv) and 12(i).

Any reference to “solid, impermeable” in relation to the base of the NT array refers to the base of the NT array being substantially solid and impermeable throughout its width, with essentially no porosity such that the contents loaded into the cavity cannot drain away or cannot be accessed through the base. It will be understood that the NTs attached at one end to the solid impermeable base do not act as nanostraws nor act as a means for flowing biomolecules through the solid impermeable base. In contrast, the NTs of the present disclosure act as reservoirs with the solid, impermeable base as a closure at the bottom of the inner cavity of the NT. The inventors have surprisingly identified that the NTs can efficiently deliver biomolecules to cells via endocytosis as opposed to complex microfluidic integrated methods, whilst maintaining good cell viability.

The solid impermeable base and NTs may be integral with one and other. For example, the plurality of NTs and base may be provided as a single phase (e.g. the base is formed in-situ as a result of the formation of the NTs via reactive ion etching (RIE)). In one embodiment, it will be appreciated that where RIE is used to prepare the NT array, the base and NTs may be integral with each other. Alternatively, the base and NTs may be provided as separate, non-integral components, for example, where the base is of different material to that of the NTs. In this example, the plurality of NTs may be prepared separately (for example by solution phase methods) and subsequently attached to the surface of the base. The base may be formed from the same or different materials as the NTs. For example, the base may comprise silicon, silicon oxide, polystyrene, glass and/or organic or hybrid polymers (e.g. SU8 and OROMOCOMP). In other embodiments, the based can be selected from one or more of the suitable materials listed for the NTs defined below. In one embodiment, the base is silicon. In another embodiment, the substrate is glass. In yet another embodiment, the substrate is polystyrene.

The array may also be a microwell plate as described herein, wherein the plurality of NTs are attached at one end to the base of one of one or more of the wells of the microwell plate. For example, the array may be prepared by EBL and RIE to define a microwell plate comprising a plurality of NTs extending from and/or attached at one end to the base of one or more wells of the microwell plate. In this example, the NT array is integral with the microwell plate. In this example, the microwell plate may comprise a plurality of NT arrays, for example each well of the microwell plate houses a NT array.

The shape and size of the base (e.g. width, length and/or height) may be selected depending on the size of the cells being transfected. The shape and size of the base may also be selected depending on the vessel used to incubate the cells with the NT array (e.g. a petri dish or 96 well plate). As such, it will be appreciated that various base dimensions are considered. In some embodiments, the base may be rectangular, square, circular, triangular, hexagonal, or any other polygonal shape. In one embodiment, the base is rectangular or square. The base may be of any suitable dimensions. For example, the base may have an area of between about 0.01 mm² to about 100 mm². The base may have an area (e.g. the area of the surface that the NTs are attached and extend therefrom) of at least about 0.01, 0.05, 0.1, 0.2, 0.3, 0.4, 0.5, 1, 5, 10, 20, 50, or 100 mm². The base may have an area of less than about 100, 50, 20, 10, 5, 1, 0.5, 0.4, 0.3, 0.2, 0.1, 0.05, or 0.01 mm². Combinations of these area values are also possible, e.g., the base may have an area of between about 0.01 mm² to about 5 mm², for example between about 0.01 to about 1 mm². The base may have a thickness of between about 1 nm to about 1,000 nm. The base may have a thickness of at least about 1, 5, 10, 20, 50, 100, 500 or 1,000 nm. The base may have thickness of less than about 1,000, 500, 100, 50, 20, 10, 5 or 1 nm. Combinations of these thickness values are also possible, e.g., the base may have a thickness (e.g. height) of between about 1 nm to about 50 nm, for example between about 5 nm to about 20 nm. The base may have a width and/or length between about 0.01 mm to about 10 mm. The base may have a width and/or length of at least about 0.01, 0.1, 0.2, 0.5, 1, 2, 5, or 10 mm. The base may have a width and/or length of less than about 10, 5, 2, 1, 0.5, 0.2, 0.1, or 0.01 mm. Combinations of these width and/or length values are also possible, e.g., the base may have a width and/or length of between about 0.1 mm to about 5 mm, for example between about 0.2 mm to about 2 mm.

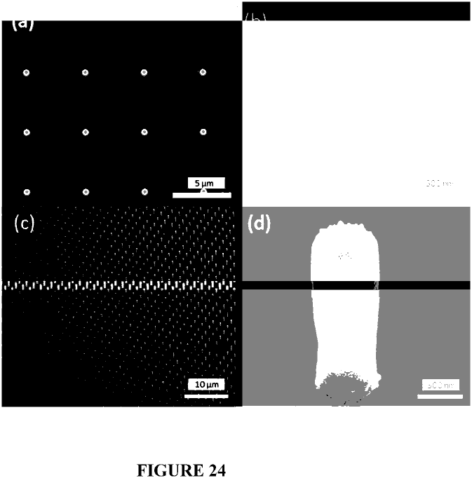

In some embodiments, the NT array comprises a plurality of NTs that are regularly spaced apart from one and other on the base, with a particular pitch, as seen in FIG. 1 by way of example for SiNTs and FIG. 24 by way of example for polymeric NTs. Alternatively, the NT array comprises a plurality of NTs that are irregularly spaced on the base, e.g. stochastic positioning. It will be appreciated that the array as described herein, is not limited to any specific spatial arrangement of the NTs.

The NTs comprise a single opening at one end of the NT (i.e. the end opposite to the end attached to the solid impermeable base, i.e. the distal end) to provide an inner cavity, see FIG. 1 (iv) and FIG. 24, (b) and (d) as examples. The inner cavity is formed by a wall boundary and the base. The inner cavity is accessible via the opening distal to the base.

The NT array has a density. The density of the NTs defines how populated the array is with NTs with reference to the number of NTs per square micrometre (µm²). In some embodiments, the average density of the NTs is at least about 0.01, 0.02, 0.03, 0.05, 0.07, 0.1, 0.2, 0.3, 0.4, 0.5, 0.6, 0.7, 0.8, 0.9, 1, 2, 3, 4, 5, 7 or 10 NTs per µm². In some embodiments, the average density of the NTs is less than about 10, 7, 5, 4, 3, 2, 1, 0.9, 0.8, 0.7, 0.6, 0.5 ,0.4, 0.3, 0.2, 0.1, 0.07, 0.05, 0.03, 0.02, or 0.01 NTs per µm². Combinations of these average density values are also possible, e.g., the average density of the NTs is between about 0.1 to about 4.0 NTs per µm², between about 0.1 to about 0.1 NTs per µm², or between about 0.1 to about 0.5 NTs per µm². In some embodiments, the NTs have a low density, for example between about 0.05 to about 1 NTs per µm², preferably between about 0.1 to about 0.5 NTs per µm².

Related to density, the NTs have a pitch, which is the average distance between each NT in the array. The average pitch of the NTs may be between about 0.1 µm to about 10 µm. The average pitch of the NTs may be at least about 0.1, 0.5 1, 2, 3, 4, 5, 6, 7, 8, 9 or 10 µm. The average pitch of the NTs may be less than about 10, 9, 8, 7, 6, 5, 4, 3, 2, 1, or 0.5 µm. Combinations of these pitch values are also possible, e.g., the average pitch of the NTs may be between about 1 µm to about 5 µm. In some embodiments, the average pitch of the NTs may be at least 1 µm, which provides a low density array.

In some embodiments, the NTs may be of any suitable length which is measured as the distance from the base where it is attached to the NT to the distal opening of the NT. The NTs may have substantially the same lengths or different lengths. In some embodiments, the average length of the NTs is at least about 0.1 µm, 0.2 µm, 0.5 µm, 1 µm, 1.5 µm, 2 µm, 2.5 µm, 3 µm, 3.5 µm, 4 µm, 4.5 µm, 5 µm, 5.5 µm, 6 µm, 6.5 µm, 7 µm, 7.5 µm, 8 µm, 8.5 µm, 9 µm, 9.5 µm, or 10 µm. In some embodiments, the average length of the NTs is less than about 10 µm, 9.5 µm, 9 µm, 8.5 µm, 8 µm, 7.5 µm, 7 µm, 6.5 µm, 6 µm, 5.5 µm, 5 µm, 4.5 µm, 4 µm, 3.5 µm, 3 µm, 2.5 µm, 2 µm, 1.5 µm, 1 µm, 0.5 µm, or 0.2 µm. Combinations of these average length values are also possible, e.g., the average length of the NTs is between about 1 µm to about 10 µm, or between about 1 µm and about 8 µm, or between about 1 µm to about 5 µm. In some embodiments, the average length of the NTs is between about 2 µm to about 5 µm, for example about 2 µm.

Each NT comprises a wall defining a single opening at the other end of the NT to provide an inner cavity. The wall defines the perimeter/boundary of the NT. The wall together with the base of the array and the distal opening defines the inner cavity of the NT. The wall may be any suitable morphology. For example, the wall may be circumferential (e.g. circular or elliptical), rectangular, hexagonal, triangular or square in shape when viewed as a cross-section. Such morphologies can be readily obtained using electron beam lithography and/or reactive ion etching. In one embodiment, the wall is a circumferential wall, as seen in FIGS. 1(ii) and (iii). The wall of the NTs may have any average thickness. The NTs may have substantially the same wall thicknesses or may have different wall thicknesses. In one embodiment, the NTs have an average wall thickness of between about 10 nm to about 1,000 nm. For example, the NTs may have an average wall thickness of at least about 10, 20, 30, 40, 50, 100, 150, 200, 250, 300, 350, 400, 450, 500, 700, or 1,000 nm. The NTs may have an average wall thickness of less than about 1,000, 700, 500, 450, 400, 350, 300, 250, 200, 150, 100, 50, 40, 30, 20, or 10 nm. Combinations of these average thickness values are also possible, e.g., the NTs may have an average wall thickness of between about 10 nm to about 500 nm, for example between about 20 nm to about 400 nm, between about 20 nm to about 200 nm, between about 50 nm to about 200 nm, for example about 100 nm.

The cross-section of the NTs may have any arbitrary shape, including, but not limited to, circular, square, rectangular, or elliptical. Regular and irregular shapes are also included. The NTs may also have any suitable diameter, or narrowest cross-section dimension if the NTs are not cylindrical in shape (e.g. irregular or frustoconical morphology). The NTs may have substantially the same diameters, or may have different diameters. In some embodiments, the average diameter of the NTs is at least about 30 nm, 50 nm, 60 nm, 70 nm, 80 nm, 90 nm, 100 nm, 120 nm, 140 nm, 160 nm, 180 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm. In some embodiments, the average diameter of the NTs is less than about 1,000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 180 nm, 160 nm, 140 nm, 120 nm, 100 nm, 90 nm, 80 nm, 70 nm, 60 nm, 50 nm, or 30 nm. Combinations of these average diameter values are also possible, e.g. the average diameter of the NTs may be between about 100 nm to about 900 nm, between about 200 nm to about 800 nm, between about 300 nm to about 700 nm, or between about 400 nm to about 600 nm. In another embodiment, the average diameter of the NTs is between about 300 nm to about 600 nm, preferably about 500 nm.

The NT’s have an inner cavity. The inner cavity may have a length substantially the same as the length of the NTs, or may have length that is shorter than the length of the NTs. For example, the inner cavity may extend from the distal opening of the NT to a location near the base of the NT. Importantly, the inner cavity does not extend through the base to provide a channel or straw morphology. Thus, the cavity of the NT is only accessible via the opening and not through the base which essentially acts as a closure. In some embodiments, the average length of the inner cavity when measured from the opening of the NT may be at least about 0.1 µm, 0.2 µm, 0.5 µm, 1 µm, 1.5 µm, 2 µm, 2.5 µm, 3 µm, 3.5 µm, 4 µm, 4.5 µm, 5 µm, 5.5 µm, 6 µm, 6.5 µm, 7 µm, 7.5 µm, 8 µm, 8.5 µm, 9 µm, 9.5 µm, or 10 µm. In some embodiments, the average length of the inner cavity when measured from the opening of the NT may less than about 10 µm, 9.5 µm, 9 µm, 8.5 µm, 8 µm, 7.5 µm, 7 µm, 6.5 µm, 6 µm, 5.5 µm, 5 µm, 4.5 µm, 4 µm, 3.5 µm, 3 µm, 2.5 µm, 2 µm, 1.5 µm, 1 µm, 0.5 µm, or 0.2 µm. Combinations of these average length values are also possible, e.g., the average length of the inner cavity when measured from the opening of the NT may be between about 1 µm to about 10 µm, or between about 1 µm and about 8 µm, or between about 1 µm to about 5 µm, for example about 2 µm to about 5 µm.

The NTs may have any suitable inner cavity diameter, or narrowest cross-section inner cavity dimension if the NTs are not cylindrical in shape (e.g. irregular or frustoconical morphology). The NTs may have substantially the same inner cavity diameters, or may have different inner cavity diameters. In some embodiments, the average inner cavity diameter of the NTs is at least about 10 nm, 20 nm, 30 nm, 50 nm, 60 nm, 70 nm, 80 nm, 90 nm, 100 nm, 120 nm, 140 nm, 160 nm, 180 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm. In some embodiments, the average inner cavity diameter of the NTs is less than about 1,000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 180 nm, 160 nm, 140 nm, 120 nm, 100 nm, 90 nm, 80 nm, 70 nm, 60 nm, 50 nm, 30 nm, 20 nm, or 10 nm. Combinations of these average inner cavity diameter values are also possible, e.g. the average inner cavity diameter of the NTs may be between about 50 nm to about 900 nm, between about 50 nm to about 800 nm, between about 50 nm to about 600 nm, or between about 50 nm to about 500 nm. In another embodiment, the average inner diameter of the NTs is between about 200 nm to about 500 nm, preferably about 300 nm.

The single opening at the distal end of the NTs may have an average opening diameter similar to that of the average inner cavity diameter. In this embodiment, the average inner cavity diameters described herein may also apply for the average diameter of the opening of the NTs. In some embodiments, the average diameter of the single opening of the NTs is at least about 10 nm, 20 nm, 30 nm, 50 nm, 60 nm, 70 nm, 80 nm, 90 nm, 100 nm, 120 nm, 140 nm, 160 nm, 180 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm In some embodiments, the average diameter of the single opening of the NTs is less than about 1,000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 180 nm, 160 nm, 140 nm, 120 nm, 100 nm, 90 nm, 80 nm, 70 nm, 60 nm, 50 nm, 30 nm, 20 nm, or 10 nm. Combinations of these average diameter values are also possible, e.g. the average diameter of the single opening of the NTs may be between about 50 nm to about 900 nm, 50 nm to about 800 nm, from about 50 nm to about 600 nm, or from about 50 nm to about 500 nm, for example about 200 nm to about 500 nm.

The NTs have an inner cavity volume (V_{SiNT} = πr²h; SiNT inner radius (r); inner cavity length (h)). The average inner cavity volume of the NTs may be between about 0.01 µm³/NT to about 10 µm³/NT. The average inner cavity volume of the NTs may be at least about 0.01, 0.02, 0.04, 0.06, 0.08, 0.1, 0.15, 0.2, 0.5, 1, 2, 5, or 10 µm³/NT. The average inner cavity volume of the NTs may be less than about 10, 5, 2, 1, 0.5, 0.2, 0.15, 0.1, 0.08, 0.06, 0.04, 0.02, or 0.01 µm³/NT. Combinations of these inner cavity volumes are also possible, e.g. the average inner cavity volume of the NTs may be between about 0.01 and 1 µm³/NT.

The NT density, pitch, length, diameter, inner cavity diameter, single opening diameter and wall thickness may be varied depending on the type of cell and/or biomolecule being delivered. In other words, the geometry of the NTs are programmable depending on the cell type and/or biomolecule. For example, the dimensions of the inner cavity (e.g. the inner cavity diameter) can be varied depending on the type biomolecule being delivered to the cell.

In some embodiments, the NTs may be blunt. As used herein, the term “blunt” is understood to mean that the distal end of the NT which contacts the cell is of a certain diameter that does not result in a sharp tip. The smaller the diameter, the sharper the end becomes. For example, NTs having a diameter of greater than 200 nm or more may be considered blunt, as opposed to diameters smaller than 200 nm which may be considered sharp. The present inventors have surprisingly identified that, in some embodiments, blunt NTs with diameters larger than 200 nm (e.g. NTs having an average inner cavity diameter of between about 200 nm to about 500 nm) were still able to penetrate the cells and deliver biomolecules, whilst maintaining good cell viability.

It will be appreciated that the NTs may have a combination of any one of the lengths, diameters, inner cavity diameters, densities, pitches and single opening diameters described herein. By way of example only, the NTs may have i) an average density between about 0.1 to about 4.0 NTs per µm²; ii) an average length between about 1 µm to about 5 µm; iii) an average inner cavity diameter of between about 50 nm to about 500 nm; and/or iv) an average wall thickness of between about 20 nm to about 200 nm. In another example, the NTs may have i) an average density between about 0.1 to about 4.0 NTs per µm²; ii) an average length between about 1 µm to about 5 µm; iii) an average inner cavity diameter of between about 50 nm to about 500 nm; and iv) an average wall thickness of between about 20 nm to about 200 nm. Other combinations are also possible. In another example, the NTs may have i) an average density of between about 0.1 to about 0.5 NTs per µm²; ii) an average length of between about 2 µm to about 5 µm; iii) an average inner cavity diameter of between about 200 nm to about 500 nm; and/or iv) an average wall thickness of between about 50 nm to about 200 nm. In another example, the NTs may have i) an average density of between about 0.1 to about 0.5 NTs per µm²; ii) an average length of between about 2 µm to about 5 µm; iii) an average inner cavity diameter of between about 200 nm to about 500 nm; and iv) an average wall thickness of between about 50 nm to about 200 nm.

The array may further comprise one or more other projections of different morphology, such as solid, non-hollow nanowires or nanopillars (as opposed to NTs comprising a wall, opening and inner cavity). For example, during the manufacture of the NT array (such as via RIE), one or more nanowires may form at one or more positions on the base/substrate due to a defect with the patterned masking/resist used to define the NT morphology to be etched, see for example FIG. 24. Therefore, it is understood that where an array comprises other projections (such as nanowires), these projections are not considered NTs. In an embodiment, the array may comprise both a plurality of NTs and non-nanotube projections (such as a plurality of nanowires). For example, the array may comprise both a plurality of NTs and nanowires, which may be arranged on the surface of the base in an ordered or random fashion. In this embodiment, the array still comprises a plurality of NTs.

A top-down process that involves the patterning of predefined structures (e.g. such as nanorings) from a supporting substrate may be used to obtain the NT array comprising the plurality of NTs attached to the surface of a base. As a non-limiting example, the NT array may be formed by a combination of direct electron beam lithography (EBL) and deep reactive ion etching (DRIE) which can yield NT arrays with controlled geometry at predefined locations on the surface of the base. In some embodiments, this integrated approach provides efficient control over the NT etching site locations where the spacing, height, and inner diameter of NT are independently controlled. In this example, using EBL and DRIE, the sites where the NTs are defined by a patterned mask which is subsequently etched to develop the patterned sites into three-dimensional NTs. An example of suitable EBL and DRIE conditions to prepare the NT array are outlined in the examples.

Methods for patterning the mask (such as a nanoring mask) include, but are not limited to, photolithography, electron beam lithography, and nanosphere lithography casting. For example, a suitable resist may be spin coated onto a substrate and subsequently loaded into an electron beam lithography (EBL) system to form a patterned soft-mask to define the NT wall thickness and overall cross-section morphology and diameter. Deep reactive ion etching (DRIE) may be then used to obtain a plurality of silicon NTs which uses alternate cycles of passivation (e.g. O₂ passivation) and etching steps (e.g. SF₆ etching) of the patterned soft mask substrate in order to obtain a plurality of silicon NTs on the surface of the substrate. Here, the non-masked area of the substrate is etched away, and the masked area of the substrate is preserved thus forming the plurality of NTs. It will be appreciated that where DRIE is used, the substrate and silicon NTs are integral with each other. By varying the masking, passivation and etching steps, control over the geometrical parameters (e.g. the diameter, density, length, inner cavity diameter and single opening diameter) of the silicon NTs can be varied. Alternatively, the NT array may be created via integrating and combining bottom-up and top-down approaches - such as colloidal lithography, metal-assisted chemical etching, and chemical vapour deposition. The NTs may also be prepared separately and subsequently attached to the surface of the base.

In a further aspect or embodiment, there is provided a method of delivering a biomolecule to a cell, the method comprising a) combining a cell suspension containing cells with a nanotube (NT) attached at one end to a solid impermeable base, wherein the NT comprises a wall defining a single opening at the other end of the NT to provide an inner cavity, the inner cavity of the NT is loaded with a solution comprising the biomolecule, and b) incubating the cell suspension with the NT to deliver the biomolecule to the cells in the cell suspension.

In a further aspect or embodiment, there is provided a nanotube (NT) attached at one end to a solid impermeable base for delivering a biomolecule to a cell, wherein the NT comprises a wall defining a single opening at the other end of the NT to provide an inner cavity.

In one embodiment, the inner cavity of the NT is loaded with a solution comprising the biomolecule. In another or further embodiment, the NT has an inner cavity diameter of between about 200 nm to about 500 nm. It will be appreciated that the NT may have a length, diameter, inner cavity diameter, single opening diameter and wall thickness as described herein in relation to the plurality of NTs.

In a further aspect or embodiment, there is provided a nanotube (NT) for delivering a biomolecule to a cell, wherein the NT comprises a wall defining a single opening at least at one end of the NT to provide inner cavity and wherein the NT has an inner cavity diameter of between about 200 nm to about 500 nm. It will be appreciated that the NT may have a length, diameter, inner cavity diameter, single opening diameter and wall thickness as described herein in relation to the plurality of NTs.

In a related aspect or embodiment, there is provided a method of delivering a molecule to a cell, the method comprising: a) combining cells with a nanotube (NT) array, comprising: i) a solid base; and ii) a plurality of nanotubes (NTs) extending from the solid base; wherein each nanotube comprises a wall defining an opening at one end of the nanotube to provide an inner cavity, and the inner cavity of at least some of the nanotubes is loaded with a solution comprising the molecule, and b) incubating the cells with the nanotube array to deliver the biomolecule to at least some of the cells.

In a related aspect or embodiment, there is provided a nanotube (NT) array for delivering a molecule to a cell, the nanotube array comprising: i) a solid base; and ii) a plurality of nanotubes (NTs) extending from the solid base; each nanotube comprises a wall defining an opening at one end of the nanotube to provide an inner cavity. In one embodiment, the nanotubes have an average density of between about 0.1 to about 0.5 NTs per µm². In one embodiment, the inner cavity of at least some of the nanotubes is loaded with a solution comprising the molecule.

In a related aspect or embodiment, there is provided a method of delivering a molecule to a cell, the method comprising: a) combining a cell with a nanotube (NT) extending from a solid base, wherein the nanotube comprises a wall defining an opening at one end of the nanotube to provide an inner cavity, wherein the inner cavity of the nanotube is loaded with a solution comprising the molecule, and b) incubating the cell with the nanotube to deliver the molecule to the cell.

In a related aspect or embodiment, there is provided a nanotube (NT) extending from a solid base for delivering a molecule to a cell, wherein the nanotube comprises a wall defining an opening at one end of the nanotube to provide an inner cavity. In one embodiment, the inner cavity of the nanotube is loaded with a solution comprising the molecule. In one embodiment, the nanotube has an inner cavity diameter of between about 200 nm to about 500 nm.

### Nanotube Material

The plurality of NTs may be made of a suitable material. For example, the NTs may be formed from materials with low cytotoxicity. Suitable materials include, but are not limited to, silicon, silicon oxide, silicon nitride, silicon carbide, iron oxide, aluminium oxide, iridium oxide, tungsten, stainless steel, silver, platinum and gold. Other suitable materials include aluminium, copper, molybdenum, tantalum, titanium, nickel, tungsten, chromium or palladium. In some embodiments, the NT comprises or consists essentially of a semiconductor. Typically, a semiconductor is an element having semiconductive or semi-metallic properties (i.e. between metallic and non-metallic properties. An example of a semiconductor is silicon. Other non-limiting examples include elemental semiconductors, such as gallium, germanium, diamond (carbon), tin selenium, tellurium, boron or phosphorous. More than one element may be present in the NTs as the semiconductor, for example gallium nitride.

In a preferred embodiment, the plurality of NTs are silicon NTs. The silicon NTs can be prepared according to processes defined herein. The average density, length, diameter, inner cavity diameter, wall thickness and single opening diameter for the silicon NTs can vary and can be selected from any one of the density, length and diameter values described herein. For example, the silicon NTs may have i) an average density of between about 0.1 to about 0.5 NTs per µm²; ii) an average length of between about 2 µm to about 5 µm; iii) an average inner cavity diameter of between about 200 nm to about 500 nm; and/or iv) an average wall thickness of between about 50 nm to about 200 nm.

In one embodiment, the surface of the NTs may have a native oxide layer. By way of example, where the NTs are silicon NTs, the native oxide layer may comprising a Si-O layer. It will be appreciated that, when present, the nature of the native oxide layer will depend on the composition of NT. For example, if the NTs are silicon, then the native oxide layer if present, will be a silicon oxide (Si-O) layer.

In another embodiment, the plurality of NTs are polymeric NTs. The polymeric NTs can be prepared according to processes defined herein. The polymeric NTs may be formed from one or more polymers. For example, the polymeric NTs may be formed from two or more different types of polymers, such as 3 or more different type of polymers, such as 4 or more different types of polymers and including 5 or more different types of polymers. Any suitable polymer may be used to prepare the polymeric NTs, for example polymeric materials that low cytotoxicity. In some embodiments, the polymeric NTs are made from polystyrene, polyesters, polycarbonates, polypyrroles, hybrid ceramic based polymers or epoxy based photoresists, or a combination thereof. For example, suitable polymeric NTs may include, but are not limited to, polyester NTs, polyolefin NTs, polycarbonate NTs, polypropylene NTs, polystyrene NTs, polyvinyl NTs (such as polyvinyl chloride (PVC) NTs), polyurethane NTs, polyether NTs, polyamide NTs, polyimide NTs, or copolymer NTs such as PETG NTs (glycol-modified polyethylene terephthalate), methacrylate NTs, polythiophene NTs, polyacetylene NTs, polyaniline NTs, and polypyrrole NTs. Polyester NTs of interest may be aliphatic polyester NTs such as polyglycolide (PGA) NTs, polylactide (PLA) NTs, polyethylene adipate (PEA) NTs, polyhydroxyalkanoate (PHA) NTs, polycaprolactone (PCL) NTs, polyhydroxybutyrate (PHB) NTs, poly(3- hydroxybutyrate-co-3 -hydroxy valerate) (PHBV) NTs or aromatic polyesters NTs such as polyethylene terephthalate (PET) NTs, polybutylene terephthalate (PBT) NTs, polytrimethylene terephthalate (PTT) NTs, polytrimethylene terephthalate (PTT) NTs and polyethylene naphthalate (PEN) NTs, among other polyester polymeric NTs.

In one embodiment, the polymeric NTs are polypyrrole NTs. In another embodiment, the polymeric NTs are polystyrene NTs.

In another embodiment, the polymeric NTs are epoxy based photoresist polymeric NTs. For example, the polymeric NTs are SU-8 NTs. SU-8 derives its name from the presence of 8 epoxy groups, being the statistical average per moiety. An example of the structure of the SU-8 monomer is provided as follows:

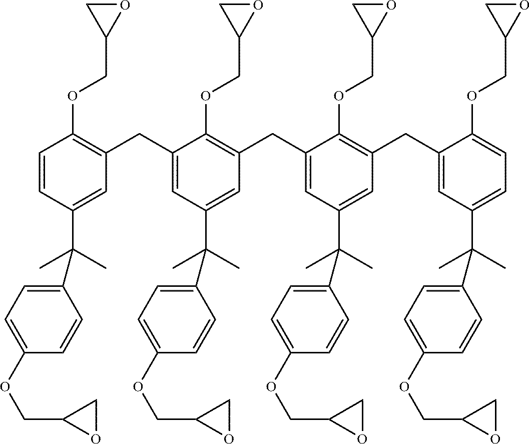 [SMILES: CC(C)(c1ccc(OCC2CO2)cc1)c1ccc(OCC2CO2)c(Cc2cc(C(C)(C)c3ccc(OCC4CO4)cc3)cc(Cc3cc(C(C)(C)c4ccc(OCC5CO5)cc4)cc(Cc4cc(C(C)(C)c5ccc(OCC6CO6)cc5)ccc4OCC4CO4)c3OCC3CO3)c2OCC2CO2)c1]

In another embodiment, the polymeric NTs are made from hybrid ceramic based polymers, e.g. (ORMOCER) NTs. For example, the ORMOCER NTs are made from the commercially available ORMOCOMP, manufactured by Microresist Technology GmbH, Berlin, Germany.

In another embodiment, the polymeric NTs may be made from polystyrene, polyesters such as Thermanox™ NTs. Thermanox™ is commercially available from ThermoFisher Scientific, and has good cell compatibility and low cytotoxicity and is used extensively in cell cultures.

The average density, pitch, length, diameter, inner cavity diameter, wall thickness and single opening diameter for the polymeric NTs can vary and can be selected from the average density, pitch, length, diameter, inner cavity diameter, wall thickness and single opening diameter values described herein. For example, the polymeric NTs may have i) an average density of between about 0.1 to about 0.5 NTs per µm²; ii) an average length of between about 2 µm to about 5 µm; iii) an average inner cavity diameter of between about 200 nm to about 500 nm; iv) an average wall thickness of between about 50 nm to about 200 nm; and or v) an average pitch of between about 0.1 µm to about 10 µm.

In one embodiment, the plurality of NTs may comprise both silicon NTs and polymeric NTs. Where the plurality of NTs comprise both silicon NTs and polymeric NTs, the average density, pitch, length, diameter, inner cavity diameter, wall thickness and opening for the silicon NTs and polymeric NTs can vary and can be selected from the average density, pitch, length, diameter, inner cavity diameter, wall thickness and single opening diameter values described herein.

### Biomolecules

The NT array may be used to deliver one or more biomolecules to the cells. The biomolecules may be delivered to the outer surface of the cell or may be delivered into the cell, for example via endocytosis following penetration of one or more NTs into the cell. The present inventors have surprisingly identified that, in some embodiments, the NT array can efficiently deliver biomolecules to cells via endocytosis whilst maintaining good cell viability.

The biomolecule may be, but is not limited to, a small biomolecule, a protein (e.g., a programmable nuclease such as Cas9, a chimeric antigen receptor (CAR), an enzyme, an antibody (e.g., monoclonal Ab)) or protein fragments such as an immunogenic peptide for use in a vaccine or an antigen binding fragment of an antibody (e.g., dAb, Fv, scFv, dimeric scFv, diabody, triabody, tetrabody, Fab, Fab′, F(ab′)2, or Fc fusions thereof), a nucleic acid (e.g., DNA, including linear and plasmid DNAs; RNA, including mRNA, siRNA, guide RNA for gene editing, dsRNA, and microRNA, PNA), nanoparticles, dyes (such as betalains e.g. betacyanins and betaxanthins), a virus, a viral particle, and polymer such as a polysaccharide (e.g. a carbohydrate). The biomolecule may also be a fluorescence-tagged protein, such as fluorescence tagged IgGs (e.g. IgG-AF647 and IgG-AF488). In some embodiments, the biomolecule may be a DNA, RNA, siRNA, miRNA a protein. Two or more biomolecules may also be delivered (e.g. co-delivered) to cells.

In an embodiment, the biomolecule is not a virus, or contained within a virus (such as a genetically modified viral genome). In this instance, the invention has significant advantages to viral mediated delivery of a biomolecule to a cell. For example, methods of delivering non-viral biomolecules to cells using the NT array disclosed herein are generally cheaper, quicker, highly scalable, and/or avoid T cell activation, and/or avoid genotoxicity due to virus-mediated transduction and/or immunogenicity due to use of virus.

The term “small biomolecule” refers to any biomolecule with a molecular weight below 1000 Da. Non-limiting examples of biomolecules that may be considered to be small biomolecules include synthetic compounds, drug biomolecules, oligosaccharides, oligonucleotides, and peptides. Combinations of one or more biomolecules described herein are also possible.

The deliberate delivery or introduction of nucleic acids into cells is typically referred to as “transfection” or “transformation”. Transfection may also refer to other methods and cell types, although other terms are often preferred. Transformation is typically used to describe non-viral DNA transfer in bacteria and eukaryotic cells such as animal cells and plant cells. Genetic material (such as supercoiled plasmid DNA or siRNA constructs), or even proteins such as antibodies, may be “transfected”. As used herein, “transfection” relates generally to delivery of a biomolecule into a cell including bacterial and eukaryotic cells.

The biomolecule may be able to modulate the expression or activity of a cellular target. The term “cellular target” refers to any component of a cell. Non-limiting examples of cellular targets include DNA, RNA, a protein, an organelle, a lipid, or the cytoskeleton of a cell. Other examples include the lysosome, mitochondria, ribosome, nucleus, or the cell membrane.

In some embodiments, the biomolecule is siRNA. siRNA, or “Small Interfering RNA,” in general is a class of double-stranded RNA biomolecules, typically 20-25 base pairs in length. siRNA plays a role in the RNA interference (RNAi) pathway, where it interferes with the expression of specific genes with complementary nucleotide sequence. Thus, for example, siRNA may have a sequence that is antisense to a sequence within a target gene. siRNA also acts in RNAi-related pathways in some cases, e.g., as an antiviral mechanism or in shaping the chromatin structure of a genome. The siRNAs typically have a structure comprising a short (usually 21-bp) double-stranded RNA (dsRNA) with phosphorylated 5′ ends and hydroxylated 3′ ends with two overhanging nucleotides. siRNAs are typically produced by the Dicer enzyme reacting with various precursor RNAs. Those of ordinary skill in the art will be able to identify siRNAs, many of which have been catalogued in publically accessible databases.

In some embodiments, the NT array described herein can be used for ex vivo gene editing. In some embodiments, the biomolecule is a programmable nuclease. As used herein, the term “programmable nuclease” relates to nucleases that can be “targeted” (“programmed”) to recognize and edit a pre-determined site in a genome.

In an embodiment, the programmable nuclease can induce site specific DNA cleavage at the pre-determined sequence or nucleic acid site. In an embodiment, the programmable nuclease may be programmed to recognize a genomic location with a DNA binding protein domain, or combination of DNA binding protein domains. In an embodiment, the programmable nuclease may be programmed to recognize a genomic location by a combination of DNA-binding zinc-finger protein (ZFP) domains. ZFPs recognize a specific 3-bp in a DNA sequence, a combination of ZFPs can be used to recognize a specific a specific genomic location. In an embodiment, the programmable nuclease may be programmed to recognize a genomic location by transcription activator-like effectors (TALEs) DNA binding domains. In an alternate embodiment, the programmable nuclease may be programmed to recognize a genomic location by one or more RNA sequences. In an alternate embodiment, the programmable nuclease may be programmed by one or more DNA sequences. In an alternate embodiment, the programmable nuclease may be programmed by one or more hybrid DNA/RNA sequences. In an alternate embodiment, the programmable nuclease may be programmed by one or more of an RNA sequence, a DNA sequence or a hybrid DNA/RNA sequence.

Programmable nucleases that can be used in accordance with the present disclosure include, but are not limited to, RNA-guided engineered nuclease (RGEN) derived from the bacterial clustered regularly interspaced short palindromic repeat (CRISPR)-cas(CRISPR-associated) system, zinc-finger nuclease (ZFN), transcription activator-like nuclease (TALEN), and argonautes.

In an embodiment, the programmable nuclease is a Clustered Regularly Interspersed Short Palindromic Repeats (CRISPR) nuclease (Barrangou, 2012). CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer).

The Type II CRISPR carries out targeted DNA double-strand break in four sequential steps (for example, see Cong et al., 2013). First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Wastson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. The CRISPR system can also be used to generate single-stranded breaks in the genome. Thus the CRISPR system can be used for RNA guided (or RNA programmed) site specific genome editing.

In an embodiment, the nuclease is a RNA-guided engineered nuclease (RGEN). In an embodiment, when the nuclease is an RGEN a guide for the nuclease, such as an RNA guide, is co-delivered to the cell with the nuclease such as in the form of a ribonucleoprotein (RNP). In an embodiment, the RGEN is from an archaeal genome or is a recombinant version thereof. In an embodiment, the RGEN is from a bacterial genome or is a recombinant version thereof. In an embodiment, the RGEN is from a Type I (CRISPR)-cas (CRISPR-associated) system. In an embodiment, the RGEN is from a Type II (CRISPR)-cas (CRISPR-associated) system. In an embodiment, the RGEN is from a Type III (CRISPR)-cas (CRISPR-associated) system. In an embodiment, the nuclease is a class I RGEN. In an embodiment, the nuclease is a class II RGEN. In an embodiment, the RGEN is a multi-component enzyme. In an embodiment, the RGEN is a single component enzyme. In an embodiment, the RGEN is CAS3. In an embodiment, the RGEN is CAS10. In an embodiment, the RGEN is CAS9. In an embodiment, the RGEN is Cpf1. In an embodiment, the RGEN is a dCAS9 or a CAS9 nickase. In a further preferred embodiment, the RGEN is a base editing enzyme or a deaminase. In a further preferred embodiment the RGEN is CAS9 coupled with a second enzyme, such as a reverse transcriptase. In an embodiment, the RGEN is targeted by a single RNA or DNA. In an embodiment, the RGEN is targeted by more than one RNA and/or DNA. In an embodiment, the RGEN is a recombinant and/or a high fidelity nuclease.

In an embodiment, the programmable nuclease may be a transcription activator-like effector (TALE) nuclease (see, e.g., Zhang et al., 2011). TALEs are transcription factors from the plant pathogen Xanthomonas that can be readily engineered to bind new DNA targets. TALEs or truncated versions thereof may be linked to the catalytic domain of endonucleases such as Fokl to create targeting endonuclease called TALE nucleases or TALENs.

In an embodiment, the programmable nuclease is a zinc-finger nuclease (ZFN). In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a FokI endonuclease. In one embodiment, the nuclease agent comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break (see, for example, US20060246567, US20080182332, US20020081614, US20030021776, WO/2002/057308, US20130123484, US20100291048 and WO/2011/017293).

In an embodiment, the programmable nuclease may be a DNA programmed argonaute (WO 14/189628). Prokaryotic and eukaryotic argonautes are enzymes involved in RNA interference pathways. An argonaute can bind and cleave a target nucleic acid by forming a complex with a designed nucleic acid-targeting acid. Cleavage can introduce double stranded breaks in the target nucleic acid which can be repaired by non-homologous end joining machinery. A DNA “guided” or “programmed” argonaute can be directed to introducing double stranded DNA breaks in predetermined locations in DNA.

In an embodiment, the biomolecule is not bound/linked to the NTs. For example, the biomolecule is not covalently attached to the surface (inner or outer surface) of the NTs and thus the surface is not “functionalised”.

The inner cavity of at least some of the NTs comprise the biomolecule, for example, the inner cavity of at least some of the NTs may be loaded with a solution comprising the biomolecule. As used herein, the term “loaded” or “loading” refers to the inner cavity of a NT being able to carrying/act as a reservoir for the biomolecule, for example, the inner cavity is at least partially filled with a solution comprising the biomolecule.

The NT array may be used to co-deliver two or more biomolecules to a cell, wherein the inner cavity is loaded with a solution comprising two or more biomolecules to co-deliver the biomolecules to the cell. The present inventors have surprisingly found that the inner cavity of the NTs can be loaded with various biomolecules which remain stable and the biomolecules do not leach out of the opening of the NTs prior to being exposed to the cells. In some embodiments, the NTs are not surface functionalised (e.g. not functionalised with one or more linkers to enhance the binding of the biomolecules).

The biomolecule may be provided in a solution which is loaded into the NTs. The solution may comprise of one or more pharmaceutically acceptable excipients, such as water or an appropriate buffer. The loading concentration of the biomolecule in the solution may at least about 0.1, 1, 2, 5, 20, 50, 100, 150, 200, 400, 600, 800, 1,000, 1,500 or 2,000 µg.mL⁻¹. The loading concentration of the biomolecule in the solution may be less than about 2,000, 1,500, 1,000, 800, 600, 400, 200, 150, 100, 50, 20, 5, 2, 1, or 0.1 µg.mL⁻¹. Combinations of these loading concentrations are also possible, e.g. the loading concentration of the biomolecule in the solution may be between about 100 µg.mL⁻¹ to about 2,000 µg.mL⁻¹.

In some embodiments, the NTs have a loading capacity (e.g. the maximum volume of solution that can be added into the inner cavity). The NTs may have an average loading capacity of between about 0.01 µm³/NT to about 10 µm³/NT. The NTs may have an average loading capacity of at least about 0.01, 0.02, 0.04, 0.06, 0.08, 0.1, 0.15, 0.2, 0.5, 1, 2, 5, or 10 µm³/NT. The NTs may have an average loading capacity of less than about 10, 5, 2, 1, 0.5, 0.2, 0.15, 0.1, 0.08, 0.06, 0.04, 0.02, or 0.01 µm³/NT. Combinations of these loading capacity values are also possible, e.g. the NTs may have an average loading capacity of between about 0.01 and 1 µm³/NT, for example about 0.1 µm³/NT.

In some embodiments, the NTs are incubated with the biomolecule for at least about 0.5 h (30 minutes), 1 h, 2 h, 3 h, 4 h, 5 h, 6 h ,7 h, 8 h, 9 h, 10 h, 15 h, 20 h, or 24 h. In other embodiments, the NTs are incubated with the biomolecule for less than about 24 h, 20 h, 15 h, 10 h, 9 h, 8 h, 7 h, 6 h, 5 h, 4 h, 3 h, 2 h, 1 h, or 0.5 h. Combinations of these incubation times are also possible, e.g. the NTs are incubated with the biomolecule for about between about 30 minutes (0.5 h) to about 6 h, about 30 minutes to about 2 h, for example about 1 h.

In some embodiments, the NTs are incubated with the biomolecule at a temperature of at least about 1, 5, 10, 15, 20, 25, 30, 35, or 40° C. In other embodiments, the NTs are incubated with the at a temperature of less than 40, 35, 30, 25, 20, 15, 10, 5, or 1° C. Combinations of these incubation temperatures are also possible, e.g., the NTs are incubated with the biomolecule at a temperature of between about 10 to 30° C., or between about 20 to 30° C., preferably about room temperature (20-25° C.).

The NT array may be pre-treated prior to loading with biomolecules to enhance the hydrophilicity of the inner cavity, for example by treatment with ozone. The NT array may be pre-treated to sterilise the surface of the array prior to combining with one or more cells.

In some embodiments, the method further comprises the step of removing any excess biomolecule(s) that are not loaded into the NT cavity. Various methods of removing excess biomolecules from the array are known in the art and include aspiration, rinsing/washing, etc.

The number of biomolecules loaded into the NTs can be defined in terms of a loading efficiency. The loading efficiency (%LE) is calculated according to the following formula: %LE = [(total biomolecules added to the NTs- free biomolecules not attached to the NTs)/total biomolecules added to the NTs] x 100%. In some embodiments, the biomolecules can be loaded into the NTs with a loading efficiency of at least about 40%, 45%, 50%, 55%, 60%, 65%, 70%, 75%, 80%, 85%, 90%, 95% or 100%. Combinations of these loading efficiency values are also possible. For example, in some embodiments, the biomolecules can be loaded into the NTs with a loading efficiency of about 40% to about 95%, about 60% to about 90%, or about 75% to about 85%.

In some embodiments, the biomolecule is delivered to the cells via endocytosis. In some embodiments, the biomolecule is delivered from the inner cavity of at least some of the NTs to at least some of the cells via endocytosis. As used herein, “endocytosis” refers to the cellular process in which substances, such as biomolecules, are brought into the cell. The biomolecule to be internalized is surrounded by an area of cell membrane, which then buds off inside the cell to form a vesicle containing the ingested material. Endocytosis includes pinocytosis and phagocytosis.

### Cells

Cells, for example as cell suspensions, are combined with the NT array. The present inventors have identified the required design parameters of NT arrays (range of diameters, heights, and densities) to allow for high-throughput delivery of biomolecules to cells.

The cells may be prokaryotic cells or eukaryotic cells. The cells may be from a single-celled organism or a multi-celled organism. In some cases, the cells are genetically engineered, e.g., the cells may be chimeric cells. The cells may be bacterial, fungi, plant, or animal cells, etc. The cells may be from a human or a non-human animal or mammal. For instance, if the cell is from an animal, the cell may be a cardiac cell, a fibroblast, a keratinocyte, a hepatocyte, a chondrocyte, a neural cell, an osteocyte, an osteoblast, a muscle cell, a blood cell, an endothelial cell, a stem cell, an immune cell (e.g., a T-cell, a B-cell, a macrophage, a neutrophil, a basophil, a mast cell, an eosinophil), etc. In some cases, the cell is a cancer cell. The cells may be non-adherent cell lines (e.g. L1.2, mouse immune B; Jurkat, human CD4⁺ T; Ramos, human immune B) or adherent cell lines (GPE86, mouse embryonic fibroblast), as well as primary cells. In an embodiment, the cell is a primary cell such as a primary immune cell or stem cell. In one embodiment, the cell is a primary immune cell, for example, a T cell. In one embodiment, the cell is an immortalised cell. The immortalised or primary cells may be an immune cell, neuron, endothelial cell, epithelial cell, or fibroblast, or mammalian cell.

In an embodiment, the cell has been modified to remove the cell wall, such as from a plant cell, e.g. a plant protoplast. Techniques for removing or permeating the wall of a cell without effecting viability are well known in the art.

As used herein, the term “immortalised cells” refers to a population of cells from a multicellular organism which would normally not proliferate indefinitely but, due to mutation, have evaded normal cellular senescence and instead can keep undergoing division.

As used herein, the term “immune cells” refer to cells of the immune system, which defend the body against disease and foreign materials. Non-limiting examples of immune cells include dendritic cells, such as bone marrow-derived dendritic cells; lymphocytes, such as B cells, T cells, and natural killer cells; and macrophages. The immune cells may, in some embodiments, be derived from bone marrow, spleen, or blood from a suitable subject. For example, the immune cells may arise from a human or a non-human mammal, such as a monkey, ape, cow, sheep, goat, horse, donkey, llama, rabbit, pig, mouse, rat, guinea pig, hamster, dog, cat, etc. In an embodiment, the immune cell is a T-cell such as a human T-cell.

As used herein, the term “stem cells” refers to clonogenic cells capable of both self-renewal and multilineage differentiation. Based on their origin, stem cells are categorised either as embryonic stem cells (ESCs) or as postnatal stem cells/somatic stem cells/adult stem cells (ASCs). Embryonic stem cells (ESCs) can be derived from embryos that are 2-11 days old called blastocysts. They are totipotent - capable of differentiating into any type of cell including germ cells. ESCs are considered immortal as they can be propagated and maintained in an undifferentiated state indefinitely.

Adult stem cells (ASCs) are found in most adult tissues. They are multipotent -capable of differentiating into more than one cell type but not all cell types. Depending on their origin, AASCs can be further classified as haemopoietic stem cells (HSCs) and mesenchymal stem cells (MSCs). HSCs can be obtained either from, for example, bone marrow, cord blood or peripheral blood. MSCs are those that originate from the mesoderm layer of the fetus and in the adult reside in a variety of tissues such as the bone marrow stem cells (BMSCc), limbal stem cells, hepatic stem cells, dermal stem cells, etc. The stem cells may be induced pluripotent stem cells.

Stem cells have also been isolated from orofacial tissues which include adult tooth pulp tissue, pulp tissue of deciduous teeth, periodontal ligament, apical papilla, and buccal mucosa.

HSCs can be divided into a long-term subset, capable of indefinite self-renewal, and a short-term subset that self-renew for a defined interval. HSCs give rise to nonself-renewing oligolineage progenitors, which in turn give rise to progeny that are more restricted in their differentiation potential, and finally to functionally mature cells including myeloid (monocytes and macrophages, neutrophils, basophils, eosinophils, dendritic cells), erythroid (erythrocytes), megakaryocytic (platelets) and lymphoid lineages (T-cells, B-cells, NK-cells).

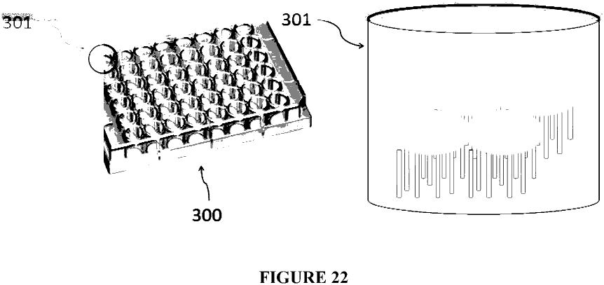

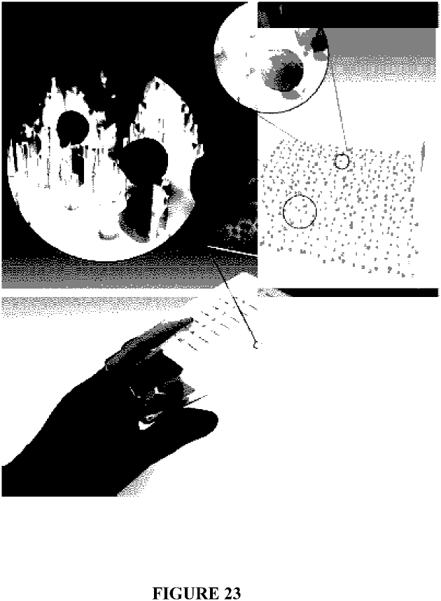

The cells/cell suspension can combined with the NT array in any manner. For example, the cells may be added onto the NTs. The cells may first be harvested and/or resuspended in a suitable medium, for example, culture medium, at a given concentration to form a cell suspension. In one embodiment, the cells in a cell suspension are combined with the NT array using, for example, a pipette, a multichannel pipette, or an automated cell plating system. In one embodiment, the cells are added to a well plate wherein one or more wells comprises one or more NT arrays comprising the plurality of NTs as described above. For example, referring to FIG. 22, one or more NT arrays comprising the plurality of NTs are placed within the wells of a multi well plate, for example a 6, 24, 48 or 96-well plate, and the cells in a cell suspension are subsequently added onto the NTs within the wells of the plate. Once combined with the NT array, the cells may be mixed with the NT array. Such mixing may include centrifugation.

In some embodiments, the number of cells per well may be at least about 10, 20, 25, 40, 50, 70, 100, 120, or 150 × 10³ cells/well. In other embodiments, the number of cells per well may be less than about 150, 120, 100, 70, 50, 40, 25, 20, or 10 × 10³ cells/well. For example, 100 × 10³ cells/well of cells (e.g., primary B or T cells) can be seeded onto NTs in a 48-well plate.

Following combining the cells with the NTs, one or more of the cells may be penetrated by at least one of the plurality of NTs. In other embodiments, the cells may rest on the surface of the opening of the NTs without being penetrated. In some cases, merely placing or plating the cells on the NTs is sufficient to cause at least some of the NTs to be inserted into the cells (i.e. penetration). For example, a population of cells suspended in medium, to make up the cell suspension, may be added to the surface of the base containing the NTs, and as the cells settle from the suspension to the surface of the base, at least some of the cells may encounter NTs, the ends comprising the single opening (e.g. the distal ends) of which may (at least in some cases) become inserted into the cells. It will be appreciated that the open ends (e.g. the distal ends) of the NT may naturally sink through the cell membranes (e.g. by gravity). The NTs may penetrate partially or completely into the cells via the cell membranes, depending on the size or dimensions of the NT and/or the size and shape of the cells. For example, the NTs may be inserted into the cytosol of a cell, or into an organelle within the cell, such as into a mitochondria, a lysosome, the nucleus, or a vacuole. In some embodiments, in a population of cells in a suspension added onto the NTs, at least about 50%, 55%, 60%, 65%, 70%, 75%, 80%, 85% or 90% of the cells may have at least one NT penetrating the cells. In other embodiments, in a population of cells added onto the NTs, less than about 90%, 85% 80%, 75%, 70%, 65%, 60%, 55% or 50% of the cells may have at least one NT penetrating the cells. Combinations of these penetration % values are also possible, e.g., in a population of cells added onto the NTs, between about 80% to about 90% of the cells may have at least one NT penetrating the cells. In some embodiments, there may be more than one NT penetrating each cell, for example on average at least about 10, 50, 100, 200, 500, or 1 × 10³ NTs.

The cells are incubated with the NTs to allow delivery of the biomolecules to the cells. For example, the cells are incubated with the NTs to allow delivery of the biomolecules from the inner cavity of at least some of the NTs to at least some of the cells. For example, the cells are incubated with the NTs to allow delivery of the biomolecules from the inner cavity of at least some of the NTs into at least some of the penetrated cells. The cells and NTs may be incubated for at least about 1 h, 4 h, 6 h, 12 h, 24 h (1 day), 2 days, 3 days, 4 days, or 5 days. The cells and NTs may be incubated for less than about 5 days, 4 , days, 3 days, 2 days, 24 h (1 day), 12 h, 6 h, 4 h or 1 h. Combinations of these incubation times are also possible, e.g. the cells and NTs are incubated for between about 1 to 6 h, e.g. about 2 h. In some embodiments, the cells and NTs are incubated at a temperature between about 30 to about 40° C., preferably approximately 37° C., or other temperatures suitable for the cell type. In some embodiments, the cells and NTs are incubated in a humidified 5% CO₂ atmosphere.

In addition, it should be noted that in some embodiments, the cells may be cultured using any suitable cell culturing technique, e.g., before or after insertion of the NTs. For example, mammalian cells may be cultured at 37° C. in a humidified 5% CO₂ atmosphere in appropriate cell medium.

In some embodiments, following incubation, the percentage of cells having biomolecules delivered to them may be at least 30%, 35%, 40%, 45 %, 50%, 55%, 60%, 65%, 70%, 75%, 80%, 85%, 90% or 95% of the total number of cells added to the NTs. Combinations of these delivery percentages are also possible, e.g., the percentage of cells having biomolecules delivered to them may be between about 30% to about 40% of the total number of cells added to the NTs, between about 40% to about 80% of the total number of cells added to the NTs, or between about 70% to about 80% of the total number of cells added to the NTs. In the context of the present disclosure, “high delivery efficiency” is understood to mean that the percentage of cells having biomolecules delivered to them may be about at least about 30% of the total number of cells added to the NTs.

In one embodiment, the method further comprises the step of centrifuging the cells and the NTs. The inventors have surprisingly identified that centrifuging the cells and the NTs not only facilitates the interaction between the cells and the NTs, but also provides an external force which allows for enhanced penetration of the cells through the cell membrane and even the nucleus or intracellular organelles. Such assisted penetration mediated by the application of an external force (e.g. centrifugation), can improve control and reliability of biomolecule delivery by increasing membrane permeability. In addition, the inventors have surprisingly identified no significant difference in cell viability before and after centrifugation. In some embodiments, this enhanced penetration via centrifugation may result in enhanced intracellular delivery of biomolecules via endocytosis triggered by the cell membrane curvature. Further advantages to the method may be provided by addition of a centrifugal force to the NTs and cells, this can reduce incubation time required to deliver the biomolecules into cells and/or result in higher % uptake of biomolecules in cells without significant impact on cell viability.

In some embodiments, the cells and NTs are centrifuged for at least about 1, 2, 3, 4, 5, 6, 7, 8, 9, 10, 12, 15, 20, 25, or 30 minutes. In other embodiments, the cells and NTs are centrifuged for less than about 30, 25, 20, 15, 12, 10, 9, 8, 7, 6, 5, 4, 3, 2, or 1 minute. Combinations of these centrifuge times are also possible, e.g., the cells and NTs are centrifuged for between about 10 minutes to about 20 minutes, for example about 15 minutes. The cells and NTs may be centrifuged at a temperature of at least 30° C., preferably about 32° C., or other temperatures suitable for the cell type. The cells and NTs may be centrifuged at a speed of about 200 × gravity to about 300 × gravity, e.g. about 250 × gravity. In other embodiments, the cells and NTs may be centrifuged at a speed of at least about 700, 800, 900, 1000, 1100, 1200, 1300, 1400, or 1500 RPM.

The skilled person will appreciate that the molecular delivery efficiency to different cell types can be manipulated by varying the centrifugation time and/or speed. For example, a centrifugation time of about 15 minutes at 250 × g shows unexpectedly high delivery efficiency to human primary immune cells.

The cells can be cultured while still attached to the NTs. Alternatively, the cells can be detached from the NTs. Such detachment processes are known in the art, and include trypsinisation or gentle pipetting. For example, the cells can be detached and harvested from the NTs after 2 h, 6 h, 12 h and 24 h of incubation. In some embodiments, following performing a method of the invention the cells are harvested and cultured to expand the cell population. For example, the cells may be cultured for 1, 2 or 3 days.

In an embodiment, the biomolecule is a polynucleotide which becomes integrated into the genome of the cell. In an alternate embodiment, the biomolecule modifies the genome of the cell such as when using a programmable nuclease.

In some embodiments, the cells are transfected with foreign genetic material. In some embodiments the cells are transiently transfected and express the foreign gene but do not integrate it into their genome. Thus the new gene will not be replicated. These cells express the transiently transfected gene for a finite period of time, usually several days, after which the foreign gene is lost through cell division or other factors. In other embodiments, stably transfected cells are generated. To accomplish this, a marker gene is typically co-transfected, which gives the cell some selectable advantage, such as resistance towards a certain toxin. Some (very few) of the transfected cells will, by chance, have integrated the foreign genetic material into their genome. If the toxin is then added to the cell culture, only those few cells with the marker gene integrated into their genomes will be able to proliferate, while other cells will die. After applying this selective stress (selection pressure) for some time, only the cells with a stable transfection remain and can be cultivated further.

Contemplated uses of the disclosed system and methods include, for example, the production of cells for therapeutic use, production of cells that may produce a recombinant product which may be a therapeutic, nutraceutical or food, for assaying the effect (intra and/or intercellular effect) of a biomolecule (e.g., candidate therapeutic), for assaying dose-response of a biomolecule, etc. The skilled person will appreciate that the intended use of the cells determines whether the cells need to be detached from the NTs.

The system and delivery methods of the disclosure can be used to generate antigen-specific CAR⁺ T cells. In certain embodiments, the system disclosed herein is used to mediate gene transfer of CARs into primary immune T cells. A wide range of CAR constructs and expression vectors for the same are known in the art. Using the NT array and methods disclosed herein, T cells can be genetically engineered to produce CARs on their surface. Advantageously, the T cells do not need to be first activated, for example, using low-level activation by phytohemagglutinin (PHA) prior to transfection. Further, short term culture (6-12 h) is sufficient for cells to uptake the CAR gene with high efficiency.

CARs encode for transmembrane chimeric biomolecules with dual function: (a) immune recognition of tumor antigens expressed on the surface of tumor cells; (b) active promotion and propagation of signaling events controlling the activation of the lytic machine. CARs comprise an extracellular domain with a tumor-binding moiety, typically a single-chain variable fragment (scFv), followed by a hinge/spacer of varying length and flexibility, a transmembrane (TM) region, and one or more signaling domains associated with the T-cell signaling. The first generation CARs are equipped with the stimulatory domain of the ζ-chain; in the second generation CARs, the presence of costimulatory domains (CD28) provides additional signals to ensure full activation; in the third generation CARs an additional transducer domain (CD27, 41-BB or OX40) is added to the ζ-chain and CD28 to maximize strength, potency, and duration of the delivered signals; the fourth generation CARs include armored CARs, engineered to synthetize and deliver interleukins. Armored CARs combine the CAR functional activities with the secretion of IL-2 or IL-12 expressed as an independent gene in the same CAR vector.

A chimeric antigen receptor (CAR) recognizes cell-surface tumor-associated antigen independent of human leukocyte antigen (HLA) and employs one or more signaling biomolecules to activate genetically modified T cells for killing, proliferation, and/or cytokine production. Adoptive transfer of T cells expressing CAR has shown promise in multiple clinical trials.

In one embodiment, cells are removed from a patient’s body (for example, blood is removed from the patient to obtain T cells) and genetically modified so that they can recognize the patient’s cancer cells (for example, transfected with gene encoding a CAR using the system and delivery methods of the present disclosure) and the modified T cells reintroduced into the patient. The modified T cells, when reintroduced into the patient’s body, multiply and attack cancer cells. In some embodiments, the modified T cells are cultured ex vivo prior to being reintroduced into the patient. Preferably, the T cells are not activated prior to introduction of the gene encoding the CAR.

In one embodiment, there is provided a population of cells comprising a molecule (e.g. a biomolecule), and/or which have been modified by the molecule, the molecule having been delivered to the population of cells, or progenitors thereof, by application and incubation on nanotube arrays.

### Kits

In another embodiment, a kit may be provided for delivering a biomolecule to a cell. The kit may include a NT array as described above.

For example, the kit may comprise a nanotube (NT) array, comprising:

i) a solid impermeable base; and ii) a plurality of nanotubes (NTs) attached at one end to the solid impermeable base; wherein each NT comprises a wall defining a single opening at the other end of the NT to provide an inner cavity.

In one embodiment, the NT array of the kit may be loaded with a solution comprising the biomolecule. For example, the inner cavity of at least some of the NTs may be loaded with a solution comprising the biomolecule.

The NTs may have i) an average density between about 0.1 to about 1.0 NTs per µm²; ii) an average length between about 1 µm to about 10 µm; iii) an average inner cavity diameter of between about 100 nm to about 500 nm; iv) an average wall thickness of between about 20 nm to about 400 nm; and/or v) an average pitch of between about 0.1 µm to about 10 µm.

In a further or another embodiment, the kit may further comprise a biomolecule. The biomolecule may be provided in a solution. In other embodiments, the kit may further comprise cells. The solution may be provided separately, or may be loaded into the inner cavity of at least some of the NTs.

It will be appreciated that embodiments described for the NTs, biomolecules, and cells earlier in relation the NT array and method of delivery a biomolecule to a cell can also provide embodiments for the NTs, biomolecules and cells that are part of the kit.

In another embodiment, the kit comprises a vessel which can house one or more NT arrays and the cells. The vessel may be a flask, multiwall plate or petri-dish. For example, the kit may comprise a well plate. The well plate may be a single well plate (e.g. a petri dish) or may be a multiwell plate comprising the NT array as discussed above. The multiwell plates may be of any size. However, in certain embodiments of the kit, the multiwell plate has the dimensions of a microwell plate, e.g., having standard dimensions (about 5 inches × about 3.33 inches, or about 128 mm × 86 mm) and/or standard numbers of wells therein. For example, there may be 6, 24, 48, 96, 384, 1536 or 3456 wells present in the multiwell plate. The multiwell plates may be fabricated from any suitable material, e.g., polystyrene, polypropylene, polycarbonate, cyclo-olefins, or the like. Microwell plates can be made by injection molding, casting, machining, laser cutting, or vacuum sheet forming one or more resins, and can be made from transparent or opaque materials. Many such microwell plates are commercially available.

In some embodiments, the multiwell plate is prepared by attaching to a bottomless multiwell plate a NT array comprising a plurality of NTs as discussed above. For example, the bottomless multiwell plate may be a commercially available bottomless microwell plate, e.g., a bottomless 384-well microwell plate. The substrate and NTs form a base of the bottomless microwell plate.

In some embodiments, the multiwell plate and the substrate may be integrated to each other by the use of a suitable adhesive. Non-limiting examples of adhesives include acrylic adhesives, pressure- sensitive adhesives, silicone adhesives (e.g., UV curable silicones or RTV silicones), biocompatible adhesives, epoxies, or the like. Non-limiting examples of biocompatible glues include, but are not limited to, Master Bond EP42HT-2ND-2MED BLACK and Master Bond EP42HT-2 CLEAR (Master Bond). The adhesive, in some cases, may be a permanent adhesive. Many such adhesives can be obtained commercially from companies such as 3M, Loctite, or Adhesives Research.

The multiwell plate and the NT array may be directly attached to each other, and/or there may be other materials positioned between the multiwell plate and the base of the NT array, for example, one or more gaskets (e.g., comprising silicone, rubber, neoprene, nitrile rubber, fiberglass, polytetrafluoroethylene, etc.). In some cases, these materials may be dimensioned and arranged to be in the same pattern as the wells (or a subset thereof) of the multiwell plate to which they are being attached.

In another embodiment, the multiwall plate is integral with the NT array.

In another embodiment, the kit comprises a petri dish comprising the NT array as described above.

### EXAMPLES

### Example 1 - Materials and Methods

### Substrate Preparation for SiNT Array Fabrication

Flat silicon wafers (4″, p-type, 3-6 Ωcm, <100>, Siltronix, France) were cleaned by sonication in 1:1 solution of ethanol:acetone for 5 min and then sonicated again in MilliQ water for 5 min. This was followed by dipping the wafers into boiling Piranha solution (3:1 H₂SO₄:H₂O₂ v/v, 75° C., Avantor Performance Materials) for 1 h to remove any organic contaminants, then washing with water and drying under a nitrogen jet.

### Making of SiNT: E-Beam Lithography (EBL)

HSQ resist (XR-1541-002, Dow Corning, USA) was spin coated onto a silicon wafer with a spin speed of 1500 rpm for 1 min. The sample was directly loaded into an electron beam lithography (EBL) system (VISTEC EBPG-5000+, Raith Company, Germany) without baking. The EBL was performed at an accelerating voltage of 100 kV with a beam current of 30 nA, using a dose of 1000 µCcm⁻². After e-beam exposure, the HSQ resist was developed using AZ 726 MIF. Development was stopped with water and samples were dried under a nitrogen jet.

### Making of SiNT: Deep Reactive Ion Etching (DRIE) of Silicon

Samples prepared by e-beam lithography were inserted into an ULVAC NLD5700 DRIE. Silicon etching was performed in a simultaneous flow of SF₆ and O₂ at a pressure of 1 Pa with Antenna RF power of 200 W and Bias RF LF power of 16 W. He pressure was set at 2000 Pa and the circulator at 20° C. The etching time was 145 s.

### Making of Polymeric NT

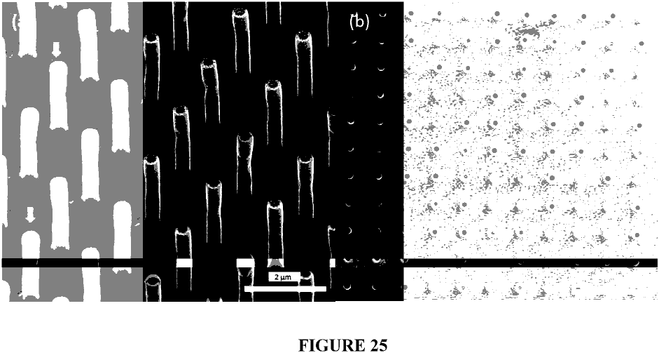

A PDMS negative master mould was first fabricated, with an inverted structure of the SiNT template. Polymeric (SU8) NTs were then fabricated via UV/thermal-nanoimprint lithography (NIL); this method utilised equipment that presses the mould onto polymer coated substrate in a vacuum, while applying UV/heat to cure and polymerise the polymer. FIG. 24 and FIG. 25 show images of several polymeric NT arrays made this way.

The experiments and analysis herein below describe the use of SiNT but could equally be replicated with Polymeric NTs.

### Calculation of SiNT Loading Capacity

Volume of internal (V_{SiNT}) = πr²h; SiNT inner radius (r) = 150 nm; internal height (h) = 1.2 µm.

### Loading of SiNTs with Bioactive Biomolecules

Prior to SiNT loading, substrates were treated with ozone for 30 min to enhance their hydrophilicity. 10 µL of fluorescence-tagged goat anti-mouse IgG (IgG-AF647, 2 µg µL⁻¹; Life Technologies), chicken anti-rabbit IgG (IgG-AF488, 2 µg µL⁻¹; Life Technologies), ssDNA-FAM (2 µg µL⁻¹; IDT), siOTP and siTOX (20 µM; Dharmacon), scramble and anti-Triobp siRNAs (20 µM; Life Technologies), Cy5-tagged GFPencoding mRNA (Cy5-mRNA-GFP, 1 mg/mL; Trilink Biotechnology), and Cas9 RNPs (complexes of Cas9 v3 protein, crRNA (non-targeting control or mouse Hprt-targeting), and tracrRNA (with or without ATTO 550 tag); IDT) were loaded onto flat Si and SiNT substrates, and incubated for 1 h at room temperature (RT). Substrates were then rinsed twice with 1 × DPBS to wash off excessive payloads. Nucleotide sequences: anti-Triobp siRNAs (5’ CCAAGCCCACAACGAUCGUTT 3’ (SEQ ID NO:1); 3′ ACGAUCGUUGUGGGCUUGGTT 5’ (SEQ ID NO:3); Life Technologies), mouse Hprt-targeting crRNA (5’ ACCTCTTAGGAGTCTAAAGTGTTTTAGAGCTATGCT 3′ (SEQ ID NO:3); IDT).

### Cell Culture

GPE86 cells (ATCC, CRL-9642) were grown and maintained in Dulbecco’s modified Eagle’s medium (DMEM, Gibco) supplemented with 10% fetal bovine serum (FBS, Gibco), 1 mM sodium pyruvate (Gibco), 2 mM L-glutamine (Gibco), 100 U mL⁻¹ penicillin (Gibco), and 100 µg mL⁻¹ streptomycin (Gibco). Cells were incubated at 37° C. with 5% CO₂.

### SiNT-Mediated Intracellular Delivery

GPE86 cells (25,000 cells/well) were seeded onto SiNT substrates (~0.3 mm²) loaded with different biomolecules (IgG-AF647, IgG-AF488, ssDNAFAM, siRNAs, or Cas9 RNPs) in a 48-well plate, in 250 µL Opti-MEM (Gibco), followed by centrifugation at 250 g, 32° C., for 15 min. After centrifugation and 2 h incubation at 37° C., 5% CO₂, substrates carrying GPE86 cells were transferred to new plates and cultured in fresh complete DMEM for a further 4 h.

For fluorescence (e.g., confocal) microscopy imaging, cells grown on substrates were rinsed with DPBS and fixed in a solution of 4% paraformaldehyde (PFA; Electron Microscopy Sciences) for 10 min, followed by permeabilization in 0.1% Triton X-100 (Sigma-Aldrich) in DPBS for 5 min at RT. After washing three times with DPBS, cells were stained with relevant fluorescence markers (Table 1) before proceeding to microscopy imaging.

For flow cytometry analysis, cells on substrates were trypsinized with 0.25% Trypsin-EDTA (Gibco), neutralized with DMEM, transferred to v-bottom 96-well plate, spun down, and washed twice with flow cytometry staining buffer (FACS buffer). Cells were stained with relevant fluorescence markers (Table 1) before proceeding to flow cytometry detection.

TABLE 1

Untagged antibodies and fluorescence markers used for confocal microscopy and flow cytometry analysis

| Fluorescence markers | λₑₓ (nm) | λₑₘ (nm) |
|---|---|---|
| Hoechst 33342 (Hoechst) (Sigma-Aldrich) | 350 | 461 |
| IgG-AF488 (Invitrogen) | 495 | 520 |
| ssDNA-FAM (IDT) | 495 | 520 |
| tracrRNA ATTO 550 (IDT) | 554 | 574 |
| AF568 Phalloidin (Thermo Fisher) | 578 | 600 |
| IgG-AF647 (Invitrogen) | 647 | 670 |
| AF647 Wheat Germ Agglutinin (WGA) (Invitrogen) | 647 | 670 |

Untagged primary antibodies
Rabbit polyclonal antibody to Tara (Triobp; Invitrogen)
Rabbit polyclonal to Caveolin-1 (CAV-1; Abcam)
Mouse monoclonal to clathrin heavy chain 1 (CHC; Invitrogen)

### Confocal Laser Scanning Microscopy Imaging

A Nikon A1R confocal laser scanning microscope system was used for fluorescence imaging. Observations were conducted at more than ten different regions on the surface of each sample at the magnification of 60 × water immersed objective lens. Images were analyzed using the Nikon NIS-Elements Advanced Research software provided by the manufacturer.

### Cell Viability Assay

The viability of cells on substrates was assayed by live-dead staining using a final concentration of 15 µg mL⁻¹ fluorescein diacetate (FDA; Sigma-Aldrich) and 10 µg mL⁻¹ propidium iodide (PI; Sigma-Aldrich) in media for 5 min at 37° C. After staining, substrates were rinsed three times with DPBS before being observed under an inverted Nikon Ti-S fluorescence microscope at the magnification of 10 × objective lens. Observations were conducted at five different locations on each substrate. All experiments were repeated at least three times.

### Internalization Test

GPE86 cells were seeded onto SiNTs pre-loaded with fluorescent internalization probe (FIP)-CF568 (λₑₓ 562 nm; λₑₘ 583 nm) and cultured for 6 h. Substrates containing cells were placed in cold DPBS with or without complementary quencher probe (Q2V1) and incubated for 30 min. Hoechst was added to stain cell nucleus 5 min before proceeding to confocal microscopy imaging. Alternatively, cells were detached from substrates after 6 h, resuspended in cold DPBS with or without Q2V1 quencher and detected by flow cytometry. DNA sequences: FIP-CF568 (5′ CF568-TCAGTTCAGGACCCTCGGCT-N3 3′ (SEQ ID NO:4)); Q2V1 (5′ AGCCGAGGGTCCTGAACTGA-BHQ2 3′ (SEQ ID NO:5)).

### Endocytosis Inhibition

GPE86 cells were treated without, and with nystatin (caveolae inhibitor, 100 µM; Sigma-Aldrich) or chlorpromazine (clathrin inhibitor, 15 µM; Sigma-Aldrich) for 1 h before transferring onto SiNTs preloaded with Cy5-mRNA-GFP. After spin and 6 h incubation, one set of cells was left on SiNTs and stained with Hoechst for confocal imaging; while the second set of cells was trypsinized and detached from SiNTs for flow cytometry detection.

### Sample Preparation for SEM Imaging

Cells grown on SiNT substrates were rinsed with 0.1 M sodium cacodylate buffer (Electron Microscopy Sciences) and fixed with 2.5% glutaraldehyde (Electron Microscopy Sciences) in 0.1 M sodium cacodylate at 4° C. overnight. Following this, substrates were washed (3 × 5 min) with chilled 0.1 M sodium cacodylate buffer and post-fixed with 1% osmium tetroxide (Electron Microscopy Sciences) in 0.1 M sodium cacodylate at RT for 1 h. After repeating the washing step, substrates were gradually dehydrated with increasing concentrations of ethanol; 50%, 70%, 90% (1 × 10 min) and 100% (2 × 10 min) at RT, and finally were critical point dried (CPD 030 Critical Point Dryer, BAL-TEC). Substrates were then mounted on SEM stubs and sputter coated with a 7 nm layer of either gold or platinum in order to increase their conductivity.

### Staining of Intracellular Compartments

The sample preparation combined heavy metal staining with resin embedding. In particular, samples were rinsed with 0.1 M sodium cacodylate buffer and fixed with 2.5% glutaraldehyde in the same buffer at 4° C. overnight. Following this, samples were washed (3 × 5 min) with chilled 0.1 M sodium cacodylate buffer and quenched with chilled 20 mM glycine solution (Sigma-Aldrich) in the same buffer for 20 min. After repeating the washing step, samples were post-fixed by combining equal volumes of 4% aqueous osmium tetroxide with 2% potassium ferrocyanide (UNIVAR) in 0.2 M sodium cacodylate buffer on ice for 1 h. Samples were then washed again (3 × 5 min) with chilled buffer and incubated with 1% tannic acid (BDH) in water at RT for 20 min. After rinsing with buffer (2 × 5 min) samples were further incubated with 2% aqueous osmium tetroxide at RT for 30 min. Following this, samples were washed (2 × 5 min) with distilled water and incubated with syringe-filtered 4% aqueous uranyl acetate (UNIVAR) at 4° C. overnight. Samples were then washed (3 × 5 min) with chilled distilled water and gradually dehydrated with increasing concentrations of ethanol; 10%, 30%, 50%, 70%, 90% and 100% (1 × 7 min) at RT. An epon resin 20 mL solution was prepared by initially mixing 12.2 g of DDSA (Dodecenyl Succinic Anhydride Specially Distilled 13710, Electron Microscopy Sciences), 4.4 g of Araldite (GY 502 10900, Electron Microscopy Sciences) and 6.2 g of Procure 812 (EMBED 812 RESIN 14900) using a mechanical stirrer. Once the solution was uniformly mixed, 0.8 mL of BDMA (N-benzyldimethylamine 11400, Electron Microscopy Sciences) was added to it while stirring. Samples were then infiltrated with increasing concentrations of the freshly prepared resin solution in 100% ethanol at RT and in a sealed container using the following ratios: 1:3 (3 h), 1:2 (3 h), 1:1 (overnight), 2:1 (3 h), 3:1 (3 h). Following this, samples were finally infiltrated with 100% resin solution overnight. Prior to polymerization at 60° C., the excess resin was drained away by mounting the samples vertically for 1 h.

### SEM Imaging

SEM imaging of both bare SiNTs and SiNT substrates with cells was performed on a Nova NanoSEM 430 (FEI). The images were taken at tilt (45°) or top views with an electron beam acceleration voltage of 3-5 kV and a current of 80 pA, while using a secondary electron detector.

### FIB Sectioning and Imaging

FIB sectioning of SiNT substrates with cells was performed using a Thermo Fischer Helios G4 UX FIB-SEM vertically and at 45° to the sample surface. Prior to FIB sectioning, the region of interest was protected from ion beam (i-beam) damage using i-beam assisted deposition of a ~0.5 µm thick platinum layer. The coating was carried out at 30 kV using i-beam current of 0.26-0.44 nA, depending on the area size. Following this, rough milling was performed at acceleration voltage of 30 kV and a current of 20 nA. The resulting cross sections were then polished with a voltage of 30 kV and a current ranging between 1.2-2.4 nA. Images were taken using an electron beam at acceleration voltage of 3 kV and a current of 200 pA using immersion mode and with a TLD detector operating in backscattered (BS) electron collection mode, at a dwell time of 5 µs and 6144 × 4096 pixel resolution. During sequential sectioning, images were taken every 20 nm using previously mentioned e-beam conditions.

### T7 Endonuclease I (T7E1) Assay

1) Target fragment amplification from genomic DNA: GPE86 cells detached from Cas9 RNP-loaded SiNTs were lysed in 50 µL Cell Lysis Buffer (Life Technologies); 2 µL of the lysate were added into AmpliTaq Gold® 360 Master Mix (Life Technologies) with primer mix (forward primer: 5′ AGGTTTCGAGCCCTGATATTCG 3′ (SEQ ID NO:6), reverse primer: 5′ ATGTGGCAAGGTCAAAAACAGT 3′ (SEQ ID NO:7); Life Technologies), followed by Polymerase chain reaction (PCR).
2) Re-annealing reaction: PCR products were denatured at 95° C. for 5 min, followed by re-annealing from 95 to 85° C. (-2° C./sec), then 85 to 25° C. (-0.1° C./sec).
3) T7E1 cleavage: Re-annealed PCR products were digested by T7E1 endonuclease (Life Technologies) for 1.5 h at 37° C.
4) DNA electrophoresis: T7E1 digested PCR products were separated by 2% agarose gel (Promega) containing GelRed (Merck) at 80 V for 1.5 h, and imaged by UVIdoc HD5 (UVItec Ltd.) system. T7E1 cleavage efficiency was calculated by the following formula: Cleavage Efficiency = 1- [(1-Fraction Cleaved) ½] Fraction Cleaved = sum of cleaved band intensities/(sum of the cleaved and parental band intensities).

### Data Processing and Statistical Analysis

Fluorescence, SEM, and DNA electrophoresis gel images were processed and analysed by Image J. The contrast and the brightness were not varied from the original pictures. Flow cytometry data were analysed with FlowJo. All statistical analysis was performed using Prism GraphPad 8. Non-parametric two-sided Mann-Whitney U-tests were performed for comparison between two groups. A one-way ANOVA was used to calculate univariate data set with more than two groups. Two-way ANOVA tests were used to calculate multivariate data set.

### Example 2 - Fabrication of SiNT Arrays

Programmable vertically aligned silicon nanotube (SiNT) arrays were fabricated by a combination of direct e-beam lithography (EBL) and deep reactive ion etching (DRIE) as described in Example 1, yielding NT arrays with controlled geometry at predefined locations on the Si surface (FIG. 1). The spacing, height and diameter were independently controllable. The resulting example SiNT arrays had dimensions of inner diameter of 300 nm, height of 2 µm and pitch of 5 µm.

### Example 3 - Loading of SiNTs With Biomolecules

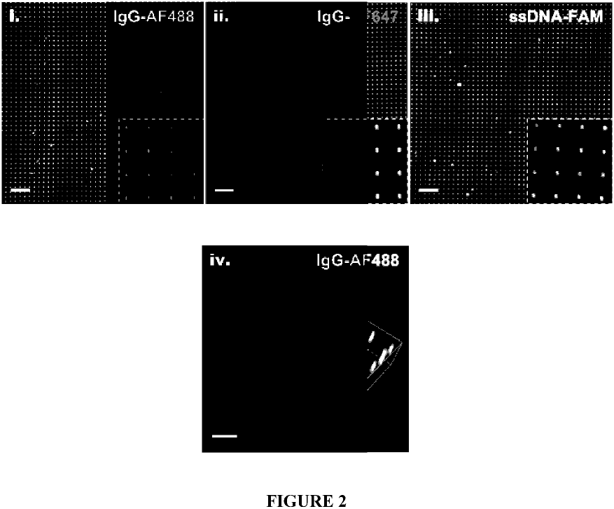

Once fabricated, the SiNT arrays of Example 2 were loaded by spotting (e.g. applying one or more droplets) a solution containing two types of fluorescently labelled bioactive materials - IgG and ssDNA. Top and 3D views of confocal microscopy images (FIG. 2) demonstrated the homogenous loading distribution of IgG-AF488, IgG-AF647 and ssDNA-FAM across the whole SiNT array.

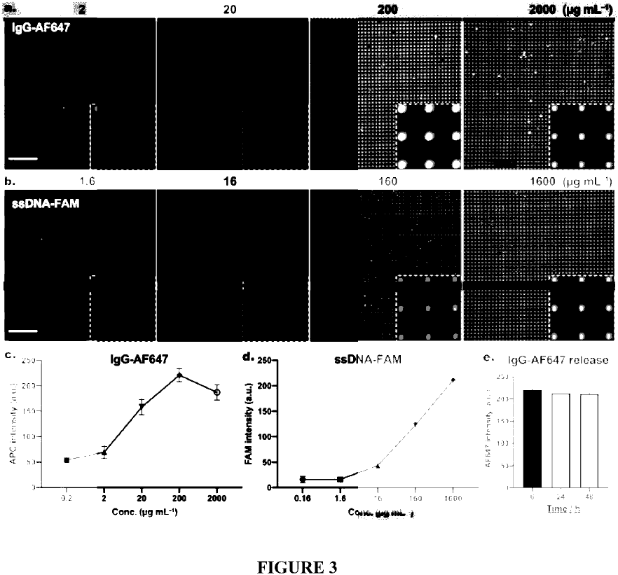

Using a serial dilution of IgGs and ssDNAs added onto SiNTs, the optimal payload concentration required to achieve maximum SiNT loading efficiency was found (FIG. 3). IgG-AF647 fluorescence intensity peaked at the loading concentration of 200 µg mL⁻¹, followed by slightly lower signal at 2,000 µg mL⁻¹ and 20 µg mL⁻¹, while a concentration of 2 µg mL⁻¹ and below generated signal close to background noise (FIGS. 3a, c). The drop in fluorescence intensity found at high IgG concentration (2,000 µg mL⁻¹) was apparently not due to self-quenching but due to aggregation of IgG and overloading of the NT lumen. Unlike IgG-AF647, the fluorescence intensity of ssDNA-FAM on SiNTs steadily increased with loading concentrations from 1.6 µg to 1,600 µg mL⁻¹ (FIGS. 3b, d). The loaded bioactive biomolecules were shown to be stabilized in SiNTs with negligible release over 48 h in DPBS (FIG. 3e).

Since SiNTs were not surface functionalized with any positively charged molecules to enhance the binding of negatively charged IgGs and ssDNAs, it is likely that the hollow structure of SiNTs itself promoted the efficient loading and retention of both proteins and oligonucleotides.

### Example 4 - SiNT-Mediated Intracellular Delivery and Internalization of Bioactive Biomolecules Into Cells

Mouse embryonic fibroblast cells (MEFs) are an important model for in vitro biological studies; but they are challenging to transfect. Although viral methods achieve high gene-delivery efficiency, challenges with cell mutagenesis and safety among others have prompted the quest for alternative non-viral gene-delivery methods in MEF cells. Given the successful and stable loading of bioactive biomolecules into SiNTs, it was investigated whether SiNTs of Example 2 could provide alternative route for non-viral intracellular delivery of biomolecules into MEFs.

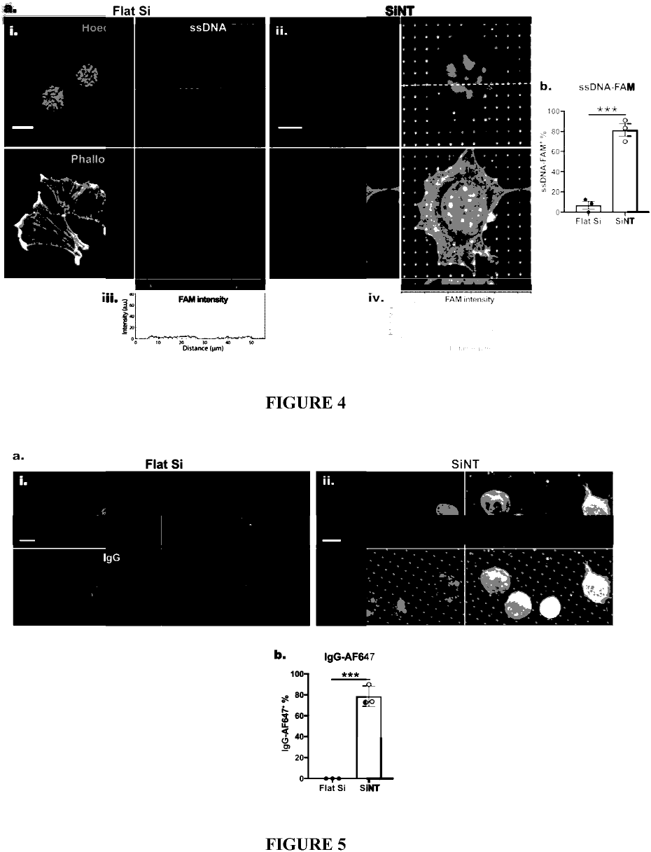

GPE86 MEF cells were seeded onto flat silicon (flat Si) and SiNT substrates that were pre-loaded with IgG-AF647 or ssDNA-FAM. To achieve rapid and optimal forceful cell-NT interfaces, external active force was applied via centrifugation at 250 g, 32° C., for 15 min. After 6 h incubation, cells were fixed on substrates and stained with Hoechst 33342 (Hoechst) and phalloidin-AF568 (phalloidin) to represent nucleus and actin cytoskeleton, respectively. Fluorescence confocal microscopy images showed the accumulation of ssDNA-FAM in the cytosol, even the nucleus, of GPE86 cells (FIG. 4aii); whereas GPE86 cells cultured on control ssDNA-coated flat Si gave undetectable FAM signal (FIGS. 4ai, iii). Side view images showed that SiNTs having intimate contact with cells lost FAM signals, while those that did not interact with cells retained FAM fluorescence (FIG. 4aiv, and FIG. 4aiii, respectively). This provided additional evidence that ssDNA-FAM was released from SiNTs into the cells during the intimate interfacing; such molecular release could be attributed to passive diffusion through disrupted membrane and/or active cellular uptake triggered by the local deformation induced by SiNTs. Statistic quantification indicated that ssDNA-FAM were delivered into 77.6 ± 1.5% of GPE86 cells on SiNT substrates, compared with 6.9 ± 3.7% of that on flat Si (FIG. 4b). Similar results were found for GPE86 cells seeded on flat Si and SiNTs pre-loaded with IgG-AF647; 81.4 ± 6.1% of cells on SiNT arrays showed IgG-AF647 inserted into the cytosol, whereas only 0.8 ± 0.4% of cells on flat Si exhibited AF647 signals (FIG. 5).

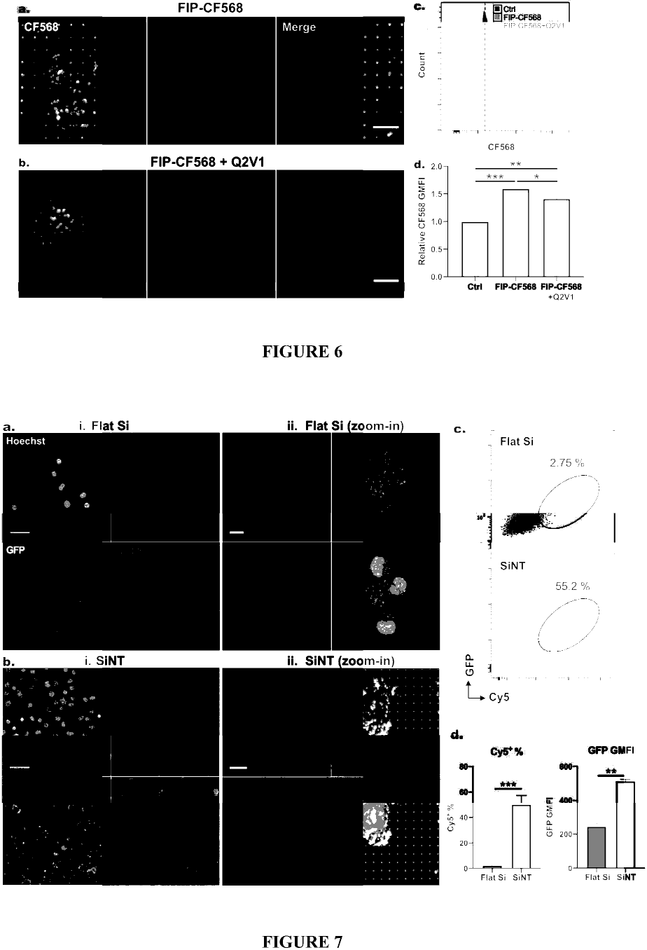

To determine whether biomolecules delivered by SiNTs gained direct cytosolic entry or remained trapped at the outer cell membrane, a fluorescent internalization probe (FIP-CF568) was employed: FIPs that were not fully internalized could be quenched instantly and permanently by Q2V1 quencher, so that only FIPs that were in the cells’ interior would emit fluorescence. Confocal images showed that while Q2V1 quenched effectively FIP-CF568 loaded in SiNTs (FIG. 6b), CF568 fluorescence could be detected in GPE86 cells incubated on SiNTs with and without subsequent treatment with Q2V1 (FIGS. 6a, b). In addition, the cells incubated on FIP-CF568 loaded SiNTs were detached after 6 h incubation and subjected to flow cytometry to analyse the CF568 fluorescence intensity within cells treated with or without Q2V1. The results demonstrated that the CF568 level of quencher-treated (FIP-CF568 Q2V1) cells was significantly higher than that of untransfected control (Ctrl) cells (FIGS. 6c, d); the slightly lower CF568 level of FIP-CF568 Q2V1 cells compared with that of cells not treated with quencher could be attributed to a small fraction of FIP-CF568 on the outer surface of the cells. Both confocal and flow cytometry findings indicate that SiNTs mediate the internalization of FIPs into GPE86 cells.

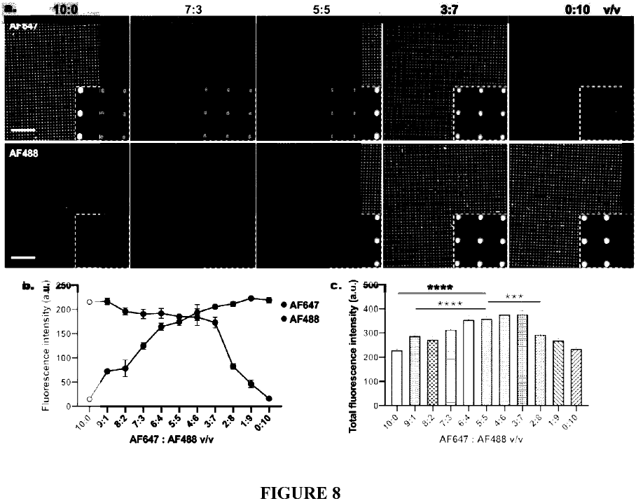

To investigate whether SiNTs-mediated internalization preserved the biological functionality of delivered biomolecules, Cy5-tagged mRNA that encodes GFP reporter (Cy5-mRNA-GFP) was used. Detection of Cy5 signal inside the cells would indicate mRNA internalization, while GFP expression would suggest the translational potency of mRNAs. GPE86 cells were added onto flat Si and SiNTs pre-loaded with Cy5-mRNA-GFP. After spin and 6 h incubation, cells were stained with Hoechst for confocal imaging. It was observed that Cy5 accumulated inside GPE86 cells on SiNTs, and these cells started to express GFP, indicating the internalization of intact mRNAs and their translation into protein product by ribosomes. (FIG. 7b). In contrast, cells on flat Si exhibited negligible Cy5 and GFP signal (FIG. 7a). Similar results were found by flow cytometry, where >50% of cells detached from mRNA-coated SiNTs contained Cy5 and exhibited higher GFP intensity compared with cells from flat Si (FIGS. 7c-d). These findings confirmed that SiNTs could mediate efficient biomolecule delivery while maintaining the biological functionality of the biomolecules. Maintaining cell viability was surprising especially as the cells were penetrated by one or more of the silicon NTs, breaching the cell membrane and in some cases penetrating the nucleus.

The capability of simultaneous delivery of multiple biomolecules into a single cell would advance cellular manipulation technology such as stem cell reprogramming, gene editing, and cancer therapy. Given the observed SiNT-mediated stable loading and intracellular delivery of IgGs and ssDNAs into GPE86 cells, it was examined whether the SiNT arrays of Example 2 could be used as a co-delivery platform.

First, IgG-AF647 and IgG-AF488 both at 200 µg mL⁻¹ concentration but with different v/v ratios were mixed, and the IgG mixture was loaded onto SiNTs. After 1 h incubation in dark at room temperature, excess payload was rinsed off. Confocal microscopy images and statistical analysis demonstrated that with decreasing AF647/AF488 input ratio (v/v), the measured intensity of AF647 decreased whereas AF488 increased (FIGS. 8a,b). The total fluorescence signal remained ≥ 200 (a.u.) across all ratios, and was maximal at AF647/AF488 ratios of 6:4, 5:5, and 4:6 (FIG. 8c), where the concentration of each IgG (AF647 or AF488) was close to 100 µg mL⁻¹. The results further confirmed that the optimal concentration for IgG loading was within 100-200 µg mL⁻¹, and indicated that SiNTs enabled biomolecule co-loading.

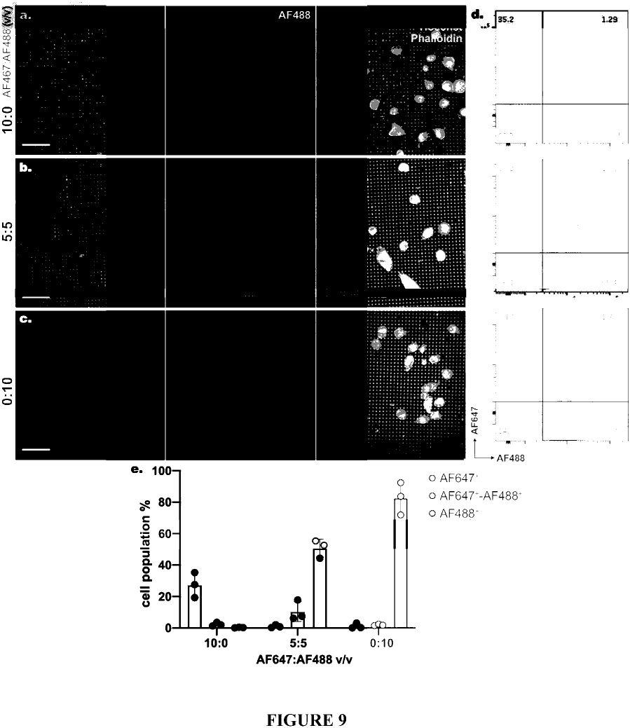

Next, GPE86 cells were seeded on SiNTs co-loaded with IgG-AF647 and IgG-AF488 (v/v, 10:0, 5:5, 0:10). After 6 h culture, samples were divided into two sets - one set had cells that remained on SiNTs for confocal microscopy imaging, the other had cells harvested from substrates by trypsinization and was analyzed by flow cytometry. The maximum intensity projection of confocal z-stack images demonstrated that the combined total signal of AF647/AF488 was reduced in SiNTs that were interacting with GPE86 cells compared with those did not have cell contact (FIGS. 9a-c). In the case where SiNTs were loaded with equal amounts of IgG-AF647 and IgG-AF488 (v/v, 5:5), the combined AF647 and AF488 fluorescence signal appeared within the cytosol (FIG. 9b) with 17.8% of AF647⁺-AF488⁺ double positive cell populations (FIG. 9e). Flow cytometry results showed that up to 83.7% of cells recovered from SiNTs with a IgG-AF647/IgG-AF488 ratio of 0:10 became AF488⁺, and 35.2% of cells contained AF647 with a IgG-AF647/IgG-AF488 ratio of 10:0 (FIG. 9d). It is not clear why the uptake efficiency of IgG-AF488 is significantly higher than that of IgG-AF647. One possible scenario could be that the origin of IgG-AF647 (goat anti-mouse) tends to trigger the protein degradation machinery such as ubiquitin-proteasome system in mouse GPE86 fibroblasts, leading to the acute and rapid degradation of IgG-AF647; while IgG-AF488 (chicken anti-rabbit) remains unaffected. Taken together, these results confirmed the use of SiNTs in co-delivering multiple biomolecules into single cells.

### Example 5 - Characterisation of the SiNT-Cell Interface

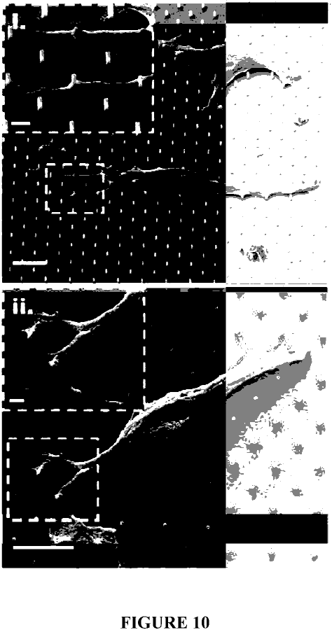

Scanning electron microscopy (SEM) images in FIG. 10 show the tilted and top view of GPE86 cells interfaced with SiNT arrays. The majority of the GPE86 cells spread and generated focal adhesions on SiNTs, with membranes reaching out to attach to the SiNTs. Long filopodial and short lamellipodia protrusions produced by GPE86 cells were also observed, which could further strengthen and promote SiNT-cell adhesion forces.

To examine if such tight interfacing would impair cell viability, a subsequent live/dead assay was performed. Fluorescein diacetate (FDA) and propidium iodide (PI) were used to stain GPE86 cells after 6 h culture on control flat Si and SiNTs with or without IgG-AF647 loading. FDA staining indicates live GPE86 cells, while the staining of impermeable dye PI indicates dead cells and damaged plasma membrane. More than 90% of cells remained viable (FDA⁺), with no PI staining, on both SiNTs and flat Si; and this was regardless of the loading of biomolecule IgG-AF647 (FIG. 11). Therefore, SiNTs did not induce significant cell death.

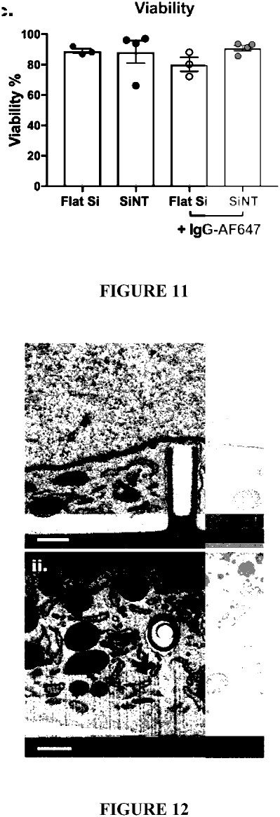

Focused ion beam (FIB)-SEM imaging data lend further support to this view. FIG. 12 showed that SiNTs induced prominent cellular and nuclear deformations of GPE86 cells, but the plasma membrane remained intact, wrapping around the SiNT elements. This type of cellular and nuclear membrane remodelling has been shown in recent studies, probing the interface between adherent cells and vertically configured nanostructures. The results from both live/dead assay and FIB-SEM imaging suggested that SiNTs represented a minimally invasive platform for intracellular delivery. Cells could be harvested after the interfacing with SiNTs and used for subsequent ex vivo and in vivo studies.

### Example 6 - Stimulation of Endocytosis Pathway by SiNTs

To determine whether clathrin-mediated endocytosis (CME) and caveolae-mediated endocytosis (CavME) could be stimulated by SiNTs, the expression and distribution of two endocytic markers, clathrin heavy chain (CHC) and caveolin-1 (CAV-1), respectively, was probed.

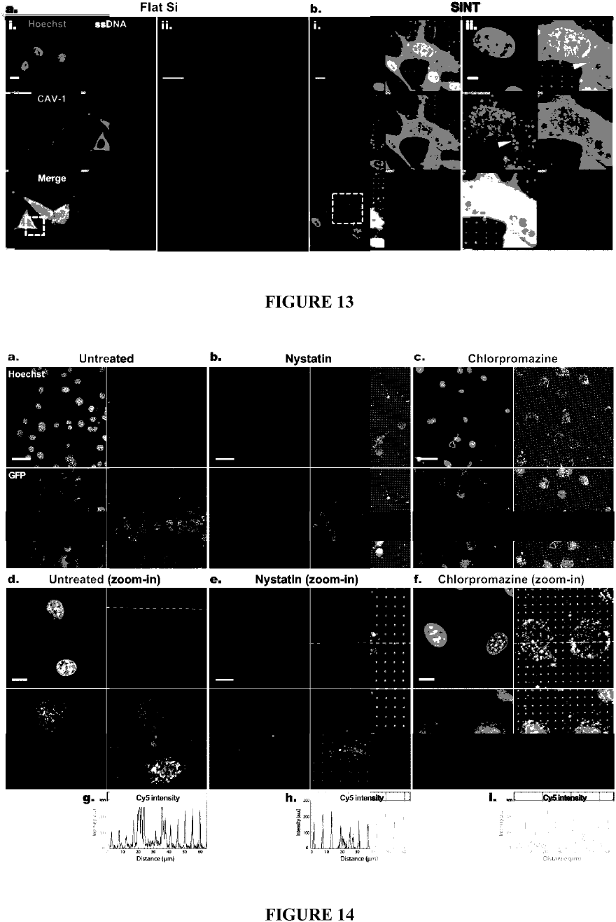

GPE86 cells were cultured for 6 h on flat Si and SiNT substrates pre-loaded with ssDNA-FAM, followed by fixation and staining with Hoechst, CAV-1, and CHC. Confocal microscopy images demonstrated the accumulation of CAV-1 at the SiNT sites (FIG. 13b), and the co-localization of CAV-1 with FAM-tagged ssDNA (indicated by arrows), which was not observed on flat Si. In contrast, the distribution of CHC was largely homogenous on both flat Si and SiNTs, and less co-localized with ssDNA compared to CAV-1, suggesting that SiNTs can directly activate endocytic processes, mainly through CavME but less likely CME.

Furthermore, the impact of endocytosis inhibition on SiNT-mediated delivery was investigated. Cells were treated with nystatin and chlorpromazine to inhibit caveolae and clathrin, respectively. After 6 h incubation on Cy-mRNA-GFP coated SiNTs, cells were stained with Hoechst and imaged by confocal microscopy. Nystatin-treated cells exhibited the lowest Cy5 signal and undetectable GFP, compared with untreated and chlorpromazine-treated cells (FIG. 14). These results indicated that the inhibition of caveolae severely impaired the cellular uptake of biomolecules from SiNTs, confirming the role of caveolae in facilitating SiNT-induced endocytosis.

### Example 7 - SiNT-Mediated siRNA Gene Knock-Down and Cas9 RNP Delivery

SiNTs were probed for their capability to achieve functional gene editing via the co-delivery of siRNAs and Cas9 ribonucleoprotein (RNP) into GPE86 cells via SiNTs.

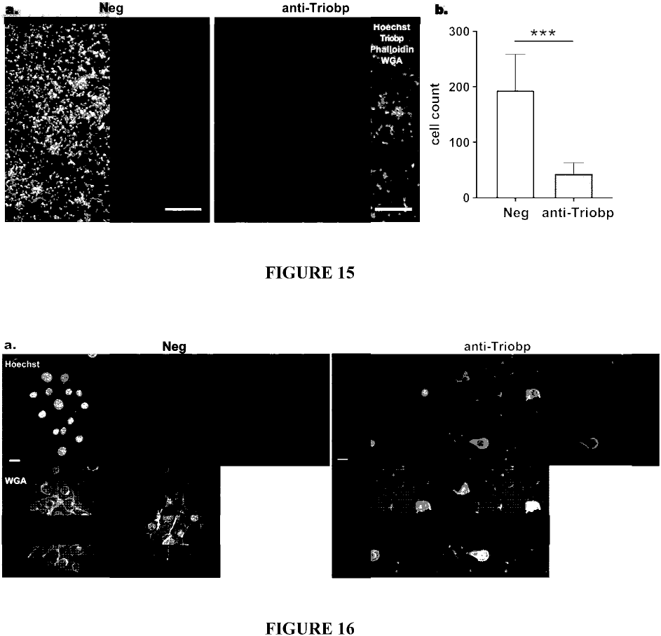

siRNAs were customized to specifically target Triobp, a F-actin bundling protein that is crucial for actin skeleton reorganization and cell migration. Knock-down of Triobp can severely impact filopodial formation and cell motility. GPE86 cells were seeded on SiNT arrays containing anti-Triobp or control scramble (Neg) siRNAs. After 48 h incubation, cells were fixed and stained with anti-Triobp, wheat germ agglutinin (WGA, stain for membrane), phalloidin, and Hoechst. The large-scan fluorescence images demonstrated the presence of even distribution and larger coverage of GPE86 cells on SiNT substrates pre-loaded with Neg siRNAs, whereas SiNTs that were loaded with anti-Triobp siRNAs showed more than four times lower cell attachment (FIG. 15).

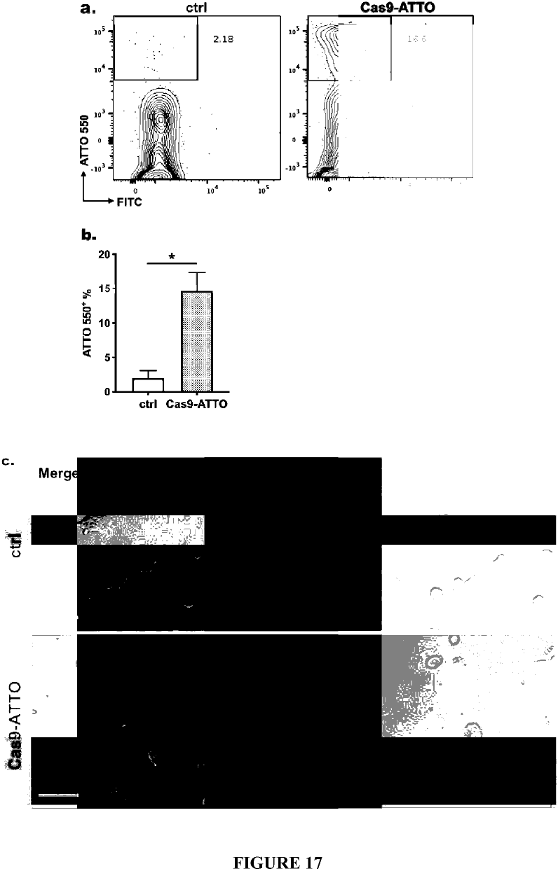

Confocal images at higher magnification (FIG. 16) provided more detailed information of nuclear morphology, Triobp expression and localization, and actin cytoskeleton of GPE86 cells on the two SiNT substrates (Neg and anti-Triobp). It was evident that cells treated with anti-Triobp siRNAs reduced dramatically the expression of Triobp, and therefore could not support the actin meshwork and filopodial formation. These phenotypical changes can significantly impact the focal adhesion and migration of GPE86 cells on SiNTs, which, at least to some extent, explains the lower cell count on anti-Triobp SiNTs. The results suggested that SiNT platforms are applicable for siRNA-mediated gene silencing.

Next, SiNT arrays were investigated for delivery of Cas9 RNPs for gene editing. Negative control CRISPR RNA (crRNA) was annealed with ATTO 550-tagged transactivating crRNA (tracrRNA-ATTO 550). The crRNA mixture was then incubated with high-fidelity S. pyogenes Cas9 nuclease V3 to generate Cas9:crRNA/tracr-ATTO 550 complex (Cas9-ATTO RNP). GPE86 cells were incubated with SiNT arrays loaded with Cas9-ATTO RNP. After 6 h culture, cells were removed from the substrates; half of the cells were analyzed by flow cytometry immediately and the second half cultured in fresh media for a further 24 h before confocal imaging. The flow cytometry results showed that 14.7 ± 1.9% of GPE86 cells were ATTO 550⁺, indicating the positive transfection of Cas9-ATTO RNP into this population, compared with the untreated control (FIG. 17a). Confocal microscopy imaging at 24 h confirmed the tracking of Cas9-ATTO RNP into the nucleus of GPE86 cells (FIG. 17b).

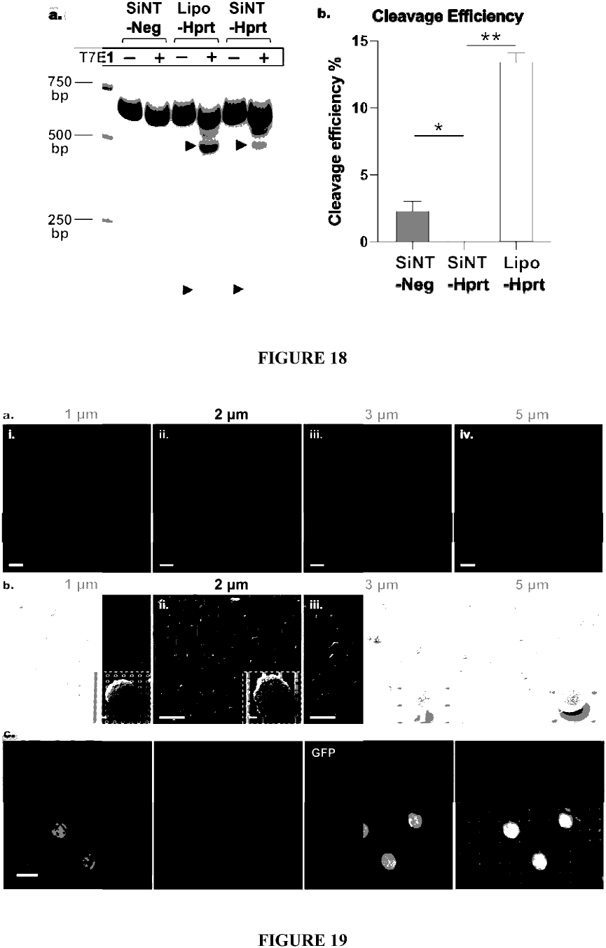

After validating the successful delivery of Cas9 RNP into cells, the gene editing efficiency was studied by delivering either a control (SiNT-Neg) or Hprt-targeted Cas9 RNP (SiNT-Hprt) into GPE86 cells using SiNTs; cells transfected with Hprt Cas9 RNP using Lipofectamine 2000 (Lipo-Hprt) served as the positive control. Using the T7 endonuclease 1 (T7E1) assay, where T7E1 cleaves DNAs at mismatched spots that can be generated by Cas9 cutting, the two cleavage bands at expected sizes (130 bp and 520 bp) were observed for cells transfected with SiNT-Hprt and Lipo-Hprt. In contrast, SiNT-Neg cells and those without T7E1 digestion did not show these two cleavage bands, but the intact DNA strand at size 650 bp (FIG. 18a). By measuring the intensity of the two cleavage products, the cleavage efficiency by SiNT-Hprt was calculated to be ~6% (FIG. 18b). The gene cleavage efficiency by SiNT-Hprt was lower than the delivery efficiency of Cas9-ATTO RNP (-14%), which might be related to sub-optimal nuclear localization and target binding.

### Example 8 - Optimisation of NT Array Geometry for Different Cell Types/Sizes

To find the optimal SiNT pitch for immune cells, primary mouse T cells were isolated using MACS depletion (Mouse Pan T kit). Cells were grown and maintained in complete RPMI (RPMI-1640 (Gibco), consisting of 10% FBS, 10 mM HEPES, 1 × non-essential amino acids solution (Gibco), 1 mM sodium pyruvate, 2 mM L-glutamine, 100 U/mL penicillin, 100 µg/mL streptomycin, and 55 µM 2-mercaptoethanol (Gibco)). Cells were incubated at 37° C. with 5% CO₂. Cells were suspended at a concentration of 0.5 million/mL (300 uL per well for 48-well plate; equivalent to 100 K -200 K cells per cm2) and cultured on SiNTs with different pitches at 1, 2, 3, and 5 µm.

From confocal and SEM (FIGS. 19a, b) showed that more T cells attached to 2 and 3 µm SiNTs, compared with 1 and 5 µm ones. The inventors then used 3 µm SiNTs to deliver Cy5-mRNA-GFP into T cells. After 6 h incubation, T cells were stained with Hoechst and imaged by confocal microscopy. Both Cy5 and GFP signal were observed inside the T cells (FIG. 19c).

### Example 9 - Polymeric NTs

The experiments and analysis herein describe the use of SiNT but could equally be replicated with polymeric NTs. Quantities of a biomolecule in an appropriate buffer would be loaded into the NT arrays (attached to a vessel as exemplified in FIG. 24) by spotting or pipetting, to achieve a concentrations of say 200 µg mL⁻¹). Primary immune cells, for example, in a cell culture suspension (say at a concentration of 0.5 million/cell) would be seeded onto the polymeric NT array and allowed to incubate for 6 h at room temperature. Centrifugation could be deployed to increase transfection if appropriate. The cells would then be harvested by trypsinisation. Several of the analytical techniques described above (flow cytometry, florescent imaging) could be used to validate the delivery of the biomolecule into the cell so as affect transfection.

### Summary of Experiments

The inventors have demonstrated the fabrication of programmable VA-SiNT arrays, and explored their use as an intracellular delivery platform for gene editing. SiNTs with loading capacity ~0.1 µm³/SiNT demonstrated loading of five biomolecule types (in particular, IgG, ssDNA, FIP, siRNA, and Cas RNP) without any prior surface functionalization and the persistence of the loaded biomolecules for more than 48 h. Adherent GPE86 cells cultured on the pre-loaded SiNT substrates exhibited a relatively high transfection efficiency within 6 h of interfacing - in particular, 78% of GPE86 cells became FAM⁺ on SiNTs loaded with ssDNA-FAM, compared to 1% of their counterparts on control flat Si.

Similar results were obtained for GPE86 cells seeded on flat Si and SiNTs pre-loaded with IgG-AF647. Both fluorescence-tagged biomolecules, IgG-AF647 and IgG-AF488, were also co-loaded into SiNTs and simultaneously delivered to GPE86 cells. SiNT-interfacing did not affect cell viability, regardless of the biomolecule loading. FIB-SEM imaging of the cell-SiNT interface revealed the absence of cell membrane penetration by the SiNTs, suggesting the involvement of an alternative mechanism in SiNT-mediated biomolecular uptake.

Confocal microscopy imaging highlighted the co-localization of endocytic biomolecules — particularly caveolin — with fluorescence-tagged biomolecules (ssDNA-FAM). Endocytosis inhibition by nystatin and chlorpromazine confirmed the involvement of caveolae rather than clathrin in SiNT-enhanced endocytosis and biomolecular delivery. SiNT-mediated siRNA gene knock-down of Triobp (F-actin binding protein) was achieved in GPE86 cells, resulting in drastically altered cell morphology and more than four times lower cell attachment compared with that on SiNTs loaded with negative control siRNAs. Cas9-ATTO RNPs were successfully (14.7% efficiency) delivered into GPE86 cells via SiNTs; 6% cleavage efficiency was obtained when using SiNTs to deliver Hprt targeting Cas9 RNP into GPE86 cells.

These results show the use of SiNTs for delivering biomolecules to cells such as for mediating ex vivo gene manipulation by transporting RGEN into cells.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The steps, features, integers, compositions and/or compounds disclosed herein or indicated in the specification of this application individually or collectively, and any and all combinations of two or more of said steps or features.

The present application claims priority from AU 2020900357 filed 10 Feb. 2020, the entire contents of which are incorporated herein by reference.

### REFERENCES

Barrangou (2012) Nature Biotechnology 30:836-838.
Cong et al. (2013) Science 339 :819-823.
Zhang et al. (2011) Nature Biotechnology 29:149-153.

1. A method of delivering a biomolecule to a cell, the method comprising:
a) combining cells with a nanotube (NT) array, comprising:
i) a solid impermeable base; and
ii) a plurality of nanotubes (NTs) attached at one end to the solid impermeable base;
wherein each NT comprises a wall defining a single opening at the other end of the NT to provide an inner cavity, and
the inner cavity of at least some of the NTs is loaded with a solution comprising the biomolecule, and
b) incubating the cells with the NT array to deliver the biomolecule to at least some of the cells.

2. The method of claim 1, wherein the method comprises co-delivering two or more biomolecules to a cell, wherein the inner cavity is loaded with a solution comprising two or more biomolecules to co-deliver the biomolecules to the cell.

3. The method of claim 1 or claim 2, wherein the method further comprises loading a solution comprising the biomolecule(s) into the inner cavity of at least some of the NTs prior to combining the cells with the NT array at step a).

4. The method of claim 3, wherein the NTs are incubated with the biomolecule(s) for about 30 minutes to about 6 h.

5. The method of any one of claims 1 to 4, wherein the biomolecule(s) are selected from one or more of a nucleic acid, a protein, a polysaccharide, or a small biomolecule, or a combination thereof.

6. The method of any one of claims 1 to 5, wherein the biomolecule(s) are selected from one or more of a DNA, RNA, or siRNA biomolecule, or a protein, or a combination thereof.

7. The method of claim 5 or claim 6, wherein the nucleic acid is a plasmid, dsDNA, mRNA, miRNA, PNA, or siRNA, or a combination thereof.

8. The method of any one of claims 5 to 7, wherein the nucleic acid encodes a chimeric antigen receptor (CAR).

9. The method of claim 5 or claim 6, wherein the protein is an antibody or a programmable nuclease.

10. The method of claim 9, wherein the programmable nuclease edits the genome of the cell.

11. The method of any one of claims 1 to 10, wherein the loading concentration of the biomolecule in the solution is between about 100 µg.mL⁻¹ to about 2,000 µg.mL⁻¹.

12. The method of any one of claims 1 to 11, wherein the cell is an adherent, non-adherent, immortalised, primary cell or stem cell.

13. The method of claim 12, wherein the immortalised cell or primary cell is an immune cell, neuron, endothelial cell, epithelial cell, or fibroblast.

14. The method of claim 13, wherein the immune cell is a T cell, B cell, dendritic cell, macrophage, or natural killer cell.

15. The method of claim 12, wherein the stem cell is an embryonic hematopoietic, mesenchymal, or induced pluripotent stem cell.

16. The method of any one of claims 1 to 15, wherein the cells are incubated with the NT array for about 1 h to about 24 h.

17. The method of any one of claims 1 to 16, wherein the cells are incubated with the NT array at a temperature of about 20° C. to about 40° C.

18. The method of any one of claims 1 to 17, further comprising the step of centrifuging the cells and NT array during the incubation at step b).

19. The method of any one of claims 1 to 18, further comprising the step of detaching the cells from the NTs after the incubation at step b).

20. The method of any one of claims 1 to 19, further comprising the step of culturing the cells.

21. The method of any one of claims 1 to 20, wherein NTs have an average density of between about 0.1 to about 4.0 NTs per µm².

22. The method of any one of claims 1 to 21, wherein the NTs have an average length of between about 1 µm to about 5 µm.

23. The method of any one of claims 1 to 22, wherein the NTs have an average inner cavity diameter of between about 50 nm to about 500 nm.

24. The method of any one of claims 1 to 23, wherein the NTs have an average wall thickness of between about 20 nm to about 200 nm.

25. The method of any one of claims 1 to 24, wherein the NTs have an average pitch of between about 0.1 µm to about 10 µm.

26. The method of any one of claims 1 to 25, wherein the NTs have a combination of any two or more of:
i) an average density of between about 0.1 to about 4.0 NTs per µm²;
ii) an average length of between about 1 µm to about 5 µm;
iii) an average inner cavity diameter of between about 50 nm to about 500 nm;
iv) an average wall thickness of between about 20 nm to about 200 nm; and
v) an average pitch of between about 0.1 µm to about 10 µm.

27. The method of any one of claims 1 to 26, wherein the NTs are silicon NTs, polymeric NTs, or a combination thereof.

28. The method of claim 27, wherein the polymeric NTs are made from polystyrene, polyesters, polycarbonates, polypyrroles, hybrid ceramic based polymers or epoxy based photoresists, or a combination thereof.

29. The method of any one of claims 1 to 27, wherein the NTs are silicon NTs.

30. The method of any one of claims 29, wherein the silicon NTs have an average density of between about 0.1 to about 0.5 NTs per µm².

31. The method of claim 29 or claim 30, wherein the silicon NTs have an average length of between about 2 µm to about 5 µm.

32. The method of any one of claims 29 to 31, wherein the silicon NTs have an average inner cavity diameter of between about 200 nm to about 500 nm.

33. The method of any one of claims 29 to 32, wherein the silicon NTs have an average wall thickness of between about 50 nm to about 200 nm.

34. The method of any one of claims 29 to 33, wherein the silicon NTs have an average pitch of between about 0.1 µm to about 10 µm.

35. The method of any one of claims 29 to 34, wherein the silicon NTs have a combination of any two or more of:
i) an average density of between about 0.1 to about 0.5 NTs per µm²;
ii) an average length of between about 2 µm to about 5 µm;
iii) an average inner cavity diameter of between about 200 nm to about 500 nm;
iv) an average wall thickness of between about 50 nm to about 200 nm; and
v) an average pitch of between about 0.1 µm to about 10 µm.

36. The method of any one of claims 1 to 35, wherein the plurality of NTs extend substantially vertically from the solid impermeable base.

37. A nanotube (NT) array for delivering a biomolecule to a cell, the nanotube array comprising:
i) a solid impermeable base; and
ii) a plurality of nanotubes (NTs) attached at one end to the solid impermeable base;
wherein each NT comprises a wall defining a single opening at the other end of the NT to provide an inner cavity.

38. The array of claim 37, wherein the inner cavity of at least some of the NTs is loaded with a solution comprising the biomolecule.

39. The array of claim 37 or claim 38, wherein NTs have an average density of between about 0.1 to about 0.5 NTs per µm².

40. The array of any one of claims 37 to 39, wherein the NTs have an average length of between about 2 µm to about 5 µm.

41. The array of any one of claims 37 to 40, wherein the NTs have an average inner cavity diameter of between about 200 nm to about 500 nm.

42. The array of any one of claims 37 to 41, wherein the NTs have an average wall thickness of between about 50 nm to about 200 nm.

43. The array of any one of claims 37 to 42, wherein the NTs have an average pitch of between about 0.1 µm to about 10 µm.

44. The array of any one of claims 37 to 43, wherein the NTs have a combination of any two or more of:
i) an average density of between about 0.1 to about 0.5 NTs per µm²;
ii) an average length of between about 2 µm to about 5 µm;
iii) an average inner cavity diameter of between about 200 nm to about 500 nm;
iv) an average wall thickness of between about 50 nm to about 200 nm; and
v) an average pitch of between about 0.1 µm to about 10 µm.

45. The array of any one of claims 37 to 44, wherein the NTs are silicon NTs, polymeric NTs, or a combination thereof.

46. The array of claim 45, wherein the polymeric NTs are made from polystyrene, polyesters, polycarbonates, polypyrroles, hybrid ceramic based polymers or epoxy based photoresists, or a combination thereof.

47. The array of any one of claims 37 to 45, wherein the NTs are silicon NTs.

48. The array of any one of claims 37 to 47, wherein the plurality of NTs extend substantially vertically from the solid impermeable base.

49. A population of cells comprising a biomolecule, and/or which have been modified by the biomolecule, the biomolecule having been delivered to the population of cells, or progenitors thereof, by application and incubation on NT arrays.

50. The population of cells of claim 49, wherein the NT array is selected from an array according to any one of claims 37 to 48.

51. The population of cells of claim 49 or claim 50, wherein the cells are primary immune cells.

52. The population of cells of any one of claims 49 to 51, wherein the cells comprise CAR⁺ T cells.

53. The population of cells of any one of claims 49 to 53, wherein the cells were not activated prior to delivery with the biomolecule.

54. The population of cells of claim 49 or claim 50, wherein the biomolecule is a programmable nuclease.

55. A kit for delivering a biomolecule to a cell, comprising:
a) a NT array according to any one of claims 37 to 48; and
b) a vessel configured to house the NT array.

56. The kit according to claim 55, wherein the vessel is a multiwell plate, a petri dish, or a flask.

57. The kit according to claim 55 or claim 56, wherein the NT array forms the base of the vessel.

58. The kit according to any one of claims 55 to 57, further comprising c) a solution comprising the biomolecule.

59. The kit according to any one of claims 55 to 58, further comprising d) a cell suspension.

60. A method of delivering a biomolecule to a cell, the method comprising:
a) combining a cell suspension containing cells with a nanotube (NT) attached at one end to a solid impermeable base,
wherein the NT comprises a wall defining a single opening at the other end of the NT to provide an inner cavity, and
the inner cavity of the NT is loaded with a solution comprising the biomolecule, and
b) incubating the cell suspension with the NT to deliver the biomolecule to the cells in the cell suspension.

61. A nanotube (NT) attached at one end to a solid impermeable base for delivering a biomolecule to a cell,
wherein the NT comprises a wall defining a single opening at the other end of the NT to provide an inner cavity.

62. The NT of claim 61, wherein the inner cavity of the NT is loaded with a solution comprising the biomolecule.

63. The NT of claim 61 or claim 62, wherein the NT has an inner cavity diameter of between about 200 nm to about 500 nm.
